(19) United States
(12) Patent Application Publication
(10) Pub. No.: US 2024/0293047 A1
(43) Pub. Date: Sep. 5, 2024

(54) METHOD AND SYSTEM FOR QUANTITATIVE PHYSIOLOGICAL ASSESSMENT AND PREDICTION OF CLINICAL SUBTYPES OF GLUCOSE METABOLISM DISORDERS

(71) Applicant: University of Virginia Patent Foundation, Charlottesville, VA (US)

(72) Inventors: BORIS P. KOVATCHEV, Charlottesville, VA (US); MARC D. BRETON, Charlottesville, VA (US); CHIARA FABRIS, Charlottesville, VA (US); DAYU LV, Charlottesville, VA (US)

(21) Appl. No.: 18/278,549

(22) PCT Filed: Feb. 23, 2022
(86) PCT No.: PCT/US22/17489

§ 371 Date: Aug. 23, 2023

Related U.S. Application Data
(60) Provisional application No. 63/152,750, filed on Feb. 23, 2021.

(30) Foreign Application Priority Data
Feb. 23, 2021 (US) 63152750

(51) Int. Cl. A61B 5/145; A61B 5/00; G16H 10/20; G16H 20/17; G16H 50/20; G16H 50/50

(52) U.S. Cl. CPC A61B 5/14532; A61B 5/7264; G16H 10/20; G16H 20/17; G16H 50/20; G16H 50/50

(57) ABSTRACT

Provided are a method, system and computer-readable storage medium for quantitative physiological assessment and prediction of clinical subtypes of glucose metabolism disorders, including but not limited to, Type 1 diabetes, obesity, pre-diabetes, gestational diabetes, or variants of Type 2 diabetes. The method allows a virtual population of in silico entities to be created, reproducing faithfully the clinical subtype distributions observed in vivo. Potential applications of this method may include, but are not limited to: (a) enabling in silico experiments assessing and/or predicting, for a real patient, treatment or intervention outcomes for a given population/clinical-subtype level; (b) pre-clinical testing of properties of new medications; (c) augmenting limited clinical trial data with in silico experiments to an extent that the domain of their validity and test corner cases may not be observed in vivo.

### CROSS-REFERENCE TO RELATED APPLICATION

This international application claims priority to and the benefit of U.S. Provisional Application No. 63/152,750 filed Feb. 23, 2021, the entire contents of which are incorporated by reference herein.

### FIELD OF THE DISCLOSURE

Disclosed embodiments relate to identification, assessment, and prediction of one or more subtypes of glucose metabolism disorders, and more specifically, to such relations as enabled by in silico modeling of variables which are then verifiable in advance of implementation of such modeling for assisting in treatment regimen.

### BACKGROUND

In connection with discussion herein, bracketed notations and/or other citation herein are to those references in the similarly entitled section hereof.

Relative to the human glucose-insulin control network, as presented in FIG. 1, blood glucose (BG) levels are raised by food containing carbohydrates, and glucose is also produced by the body (mainly by the liver), after which it is distributed and utilized through both insulin-independent (e.g., central nervous system and red blood cells) and insulin-dependent (e.g., muscle and adipose tissues) pathways. Insulin secreted by the pancreatic β-cell is the primary regulator of glucose homeostasis; it reaches the system circulation after liver degradation, and is peripherally cleared primarily by the liver. The glucose and insulin systems interact by feedback control signals. If a glucose perturbation occurs (e.g., after a meal), beta-cells secrete more insulin in direct response to increased plasma glucose concentration, or as indirect response to hormonal release from the gut, GIP and GLP-1, known as the “incretin effect” (Nauck et al 1986—[13]). In turn, insulin signaling promotes glucose utilization and inhibits glucose production to bring, rapidly and effectively, plasma glucose to its pre-perturbation level. These control interactions are usually referred to as insulin sensitivity and β-cell responsivity (Cobelli et al 2009—[5]). In pathophysiology, this feedback control is degraded. In Type 2 diabetes, the network in FIG. 1 is largely preserved, but insulin secretion is deficient relative to hepatic and peripheral insulin resistance. In particular, the incretin response is deficient (Nauck et al 1986—[14]), and this finding triggered the introduction of new classes of medications known as GLP-1 receptor agonists (incretin mimetics), and DPP-4 inhibitors (incretin enhancers) (Drucker and Nauck 2006—[7]). In Type 1 diabetes, insulin secretion is virtually absent, while glucagon secretion from the α-cell is still preserved, which removes the insulin-dependent pathways lowering BG levels, and therefore BG can only go up, leading to hyperglycemia. Thus, insulin replacement is mandatory.

A battery of counterregulatory hormones are also at work, including glucagon, epinephrine, cortisol and growth hormone, which defend on different time scales the body from life-threatening hypoglycemia. Both hypoglycemia counterregulation and insulin control are neuromediated through the brain. However, with intensive insulin treatment the counterregulatory defenses may fail, risking potentially severe hypoglycemia (White et al 1983—[17]; Cryer and Gerich 1985—[6]; Amiel et al 1988—[3]; Amiel et al 1997—[4]). Thus, in health the optimization of blood sugar control is tightly regulated by a hormonal network, and this regulation fails in diabetes at several levels: deficient insulin response from the beta cell; impaired counterregulation, and inadequate increting effect.

External regulation is therefore necessary to alleviate these deficiencies and to maintain strict glycemic control by reducing hyperglycemia without increasing the risk for hypoglycemia. BG level is both the measurable result of this optimization and the principal feedback signal to the patient, or to technology, for his/her control of diabetes (Kovatchev, 2019).

As such, it would be desirable to provide a manner of simulating verifiable clinical subtypes for use in various treatment regimen.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

Embodiments may include a method, system, and computer-readable storage medium regarding predicting one or more subtypes of glucose dysregulation, which can include generating, for an in silico population of subjects, modeling for one or more physiological variables which are respectively indicative of the one or more subtypes, determining whether one or more values of the one or more physiological variables according to the modeling are within a predetermined proximity of one or more respectively corresponding subtype variable values corresponding to the one or more subtypes when the one or more physiological variables are observed in vivo, and in response to the one or more values of the one or more physiological variables according to the modeling being determined to be within the predetermined proximity, fixing one or more parameters defining the modeling, and applying the modeling, according to the fixed one or more parameters, for a real subject to determine correspondence for the real subject to the one or more subtypes.

The one or more physiological variables may include one or more of (a) fasting glucose, (b) fasting C-peptide, (c) HOMA2-B, (d) HOMA2-IR, or (e) any combination thereof.

The one or more values of the physiological variables according to the modeling may be respectively determined based on one or more of modeling for (l) insulin secretion, (m) C-peptide secretion, (n) β-cell function, (o) insulin resistance, or (p) any combination thereof.

The one or more values of the physiological variables according to the modeling may be determined based on β-cell function in dependence on at least a value comprising (ln(fasting C-peptide level))².

The one or more subtypes may include one or more of (q) SIDD (severe insulin-deficient diabetes), (r) SIRD (severe insulin-resistant diabetes), (s) MOD (mild obesity-related diabetes), (t) MARD (mild age-related diabetes), or (u) any combination thereof.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates a schematic for the human glucose-insulin control network;

FIG. 2 illustrates a schematic for an insulin secretion model according to embodiments herein;

FIG. 3 illustrates a schematic for a C-peptide kinetics model according to embodiments herein;

FIG. 4 illustrates a linear regression of height and body weight according to data for embodiments herein;

FIG. 5 illustrates relationships among HOMA2-B, fasting glucose and C-peptide according to data for embodiments herein;

FIGS. 6A and 6B illustrate calculated HOMA2-B (normal and log) on training data according to embodiments herein;

FIGS. 7A and 7B illustrate calculated HOMA2-B (normal and log) on test data according to embodiments herein;

FIG. 8 illustrates relationships among HOMA2-B, fasting glucose and C-peptide according to modeled data for embodiments herein;

FIGS. 9A and 9B illustrate calculated HOMA2-IR (normal and log) on training data according to embodiments herein;

FIGS. 10A and 10B illustrate calculated HOMA2-IR (normal and log) on test data according to embodiments herein;

FIGS. 11A-C respectively illustrate distributions of fasting glucose (G_{b}) relative to actual, simulated, and adjusted values according to embodiments herein;

FIGS. 12A and 12B respectively illustrate HOMA2-B distributions for training and simulated data according to embodiments herein;

FIGS. 13A and 13B respectively illustrate HOMA2-IR distributions for training and simulated data according to embodiments herein;

FIGS. 14-17 respectively illustrate fasting glucose (G_{b}), HOMA2-B, HOMA2-IR, and fasting C-peptide distributions relative to training and simulated data upon adjustment of basal insulin secretion rate (SR_{b});

FIGS. 18A-33B illustrate matching for diabetes subtypes (clusters) as regards fasting glucose, fasting C-peptide, HOMA2-B, HOMA2-IR in FIGS. 18A-21B, FIGS. 22A-25B, FIGS. 26A-29B, FIGS. 30A-33B, respectively, relative to training data and simulation modeling according to embodiments herein;

FIGS. 34A-49B illustrate matching for diabetes subtypes (clusters) as regards fasting glucose, fasting C-peptide, HOMA2-B, HOMA2-IR in FIGS. 34A-37B, FIGS. 38A-41B, FIGS. 42A-45B, FIGS. 46A-49B, respectively, relative to testing data and simulation modeling according to embodiments herein; and

FIGS. 50-55 illustrate one or more environments applicable to coordination of simulation and validation of diabetes subtypes according to embodiments herein.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., a rat, dog, pig, or monkey), etc. It should be appreciated that the subject may be any applicable human patient, for example.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

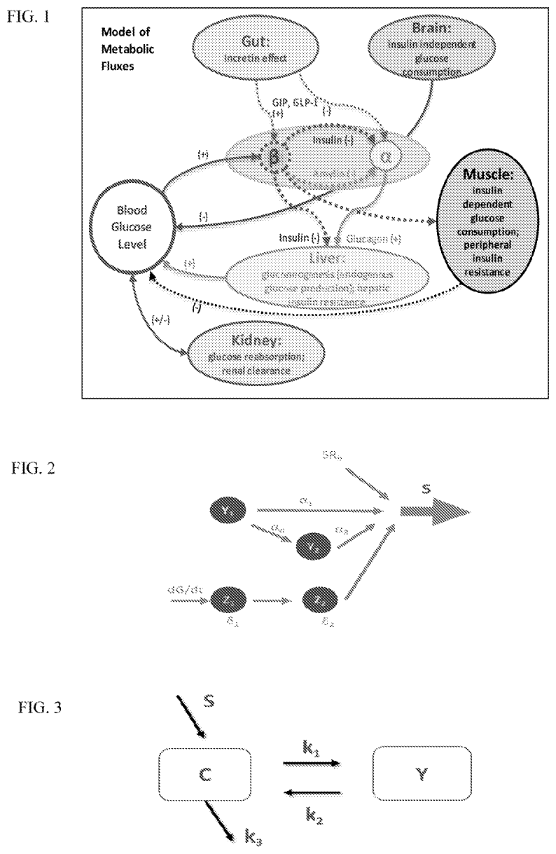

The method herein was developed using available data and now described metabolic modeling techniques to quantify the dynamics of the human gluco-regulatory network in a variety of glucose metabolism disorders, as illustrated at a high level according to FIG. 1. The metabolic parameters (which are the rate constants of internal system fluxes, e.g., glucose appearance from meals or beta-cell response) determine the magnitude and rate of the system fluxes for each individual. For example, fluxes in and out of the beta cell are virtually absent in Type 1 diabetes and can be impaired in Type 2 diabetes. This allows the metabolic system of each person to be described by a parameter vector (k₁, k₂, . . . kₛ), which uniquely identifies the metabolic phenotype for this individual.

The span of these vectors across the population and their covariance matrix determines the distribution of possible human metabolic profiles, and thereby, through sampling, can create an in silico population encompassing the variability of the human glucose dysregulation. The subtypes of glucose dysregulation applicable under the method include, but are not limited to: Type 1 diabetes, obesity, pre-diabetes, gestational diabetes, as well as variants of Type 2 diabetes identified by Ahlqvist et al, 2018: SIDD (severe insulin-deficient diabetes), SIRD (severe insulin-resistant diabetes), MOD (mild obesity-related diabetes), and MARD (mild age-related diabetes).

The in silico population can be created according to one or more devices which may be configured to implement the method to include the following:
(1) adapting an existing insulin kinetics model to encompass a variety of glucose metabolism disorders;
(2) creating a c-peptide kinetics model;
(3) modeling fasting β-cell function and fasting insulin resistance through mathematical approximations of HOMA2-B and HOMA2-IR;
(4) fine-tuning all model parameters using data from a Training Data Set (below);
(5) developing an in silico population of computer-simulated entities with physiological characteristics matching the distribution of those physiological characteristics in disease subtypes observed in vivo; and
(6) validating the in silico population using data from a Test Data Set (below).

Data: The creation of the in silico population of virtual subjects, which are stratified into identifiable subtypes and are validated against existing clinical data, used a dataset of 17092 records (all for Type 2 diabetes patients). After removing some records with missing data: Body Weight (4); Fasting C-peptide (43); Fasting plasma glucose (10), 17846 records remained, stratified in 4 clinical subtypes (clusters) of Type 2 diabetes: SIDD (severe insulin-deficient diabetes), SIRD (severe insulin-resistant diabetes), MOD (mild obesity-related diabetes), and MARD (mild age-related diabetes).

Testing and Validation: The data was randomly assigned to two groups: Training Data Set (NN-Training in the figures) (N=8924) and Test Data Set (N=8922) (NN-Testing in the figures). The Training Data Set was used to develop the method herein and match the physiological and demographic variables distributions and cluster memberships between in silico and real subjects, after which all parameters were fixed.

To validate the method on independent data, the method was then applied without further changes to the Test Data Set, whereas the proximity of the prediction generated by the method was tested/compared against the testing data, in a manner sharing similar aspects of comparisons against the training data as discussed below, in terms of the ability of the method to faithfully approximate in silico the subtypes of diabetes observed in vivo.

As a result, an in silico population of N=6156 in silico entities (subjects) was established, matching the distributions across 4 clinical subtypes of Type 2 diabetes, including several observable physiological variables, such as fasting glucose and c-peptide, as well as their derivative assessments of beta-cell function and insulin resistance, HOMA2-B and HOMA2-IR. In addition to its applicability to Type 2 diabetes, the method herein is also applicable without modification to obesity, pre-diabetes, gestational diabetes, or other glucose metabolic disorders.

### Quantitative Physiological Assessment of T2D Clinical Subtypes: Model Development and Training

Development of the above-referenced models is now discussed.

### Insulin Kinetics Model

There is a physiological variable of fasting C-peptide in the data, which necessitates the creation of a new model and simulation module describing C-peptide kinetics. Since C-peptide is produced at the same rate as insulin, the secretion rate of C-peptide is equal to that of insulin from beta-cells. A representation of insulin secretion model is given FIG. 2.

It includes two components: static (Y₁ and Y₂) and dynamic (Z₁ and Z₂) ones, which are regulated by the concentration of glucose and the changing rate of glucose respectively.

$${\dot{Y}}_{1} ( t ) = ( {α}_{1} + {α}_{d} ) \cdot ( {Y}_{1} ( t ) - max ( β \cdot ( \frac{{G}_{p} ( t )}{{V}_{g}} - {G}_{b} ) , - \frac{{SR}_{b}}{{α}_{1} + {α}_{d}} ) )$$

$${\dot{Y}}_{2} ( t ) = - {α}_{2} \cdot {Y}_{2} ( t ) + {α}_{d} \cdot {Y}_{1} ( t )$$

$${\dot{Z}}_{1} ( t ) = - {δ}_{1} \cdot ( {z}_{1} ( t ) - K \cdot \frac{{\dot{G}}_{p} ( t )}{{V}_{g}} )$$

$${Z}_{2} ( t ) = - {δ}_{2} \cdot ( {Z}_{2} ( t ) - K \cdot max ( {Z}_{1} ( t ) , 0 ) )$$

The secretion of insulin, S(t), is determined as below, where SR_{b} represents the basal secretion rate of insulin. The variable of SR_{b} may be associated with fasting C-peptide.

$$S ( t ) = {Z}_{2} ( t ) + ( {α}_{1} \cdot {Y}_{1} ( t ) + {α}_{2} \cdot {Y}_{2} ( t ) ) + S {R}_{b}$$

### C-Peptide Kinetics Model

Van Cauter et al. established a kinetics model for C-peptide distribution, providing the connection of C-peptide secretion rate to the insulin secretion rate. The model scheme is demonstrated in FIG. 3, where S represents C-peptide secretion rate (equal to insulin secretion rate); C and Y represent C-peptide amounts in plasma and peripheral tissues, respectively; k₁, k₂ and k₃ are the governing kinetic parameters.

The kinetics are given as

$$\frac{d C}{d t} = S - ( {k}_{1} + {k}_{3} ) \cdot C + {k}_{2} \cdot Y$$

$$\frac{dY}{d t} = {k}_{1} \cdot C - {k}_{2} \cdot Y .$$

To determine the kinetic parameters, variables below are defined as

$$Fraction : \frac{A}{A + B} = 0 \text{.78}$$

$$Short half ‐ life ( min ) : \frac{ln ( 2 )}{a} = 4 \text{.52}$$

$$Long half ‐ life ( min ) : \frac{ln ( 2 )}{b} = 0.14 \times Age ( yr ) + 29.2 .$$

Then, the parameters are given as

$${k}_{1} = ( a + b ) - ( {k}_{2} + {k}_{3} )$$

$${k}_{2} = \frac{( A b + B a )}{( A + B )}$$

$${k}_{3} = \frac{a b}{{k}_{2}} .$$

The volume of distribution (L) is the average of women and men determined by body surface area (BSA) such as

$$women {L}_{1} = 1.11 \times BSA + 2.04 \qquad [ 12 ]$$

$$men {L}_{2} = 1.92 \times BSA + 0.64 , where the body surface area is given as BSA ( {m}^{2} ) = L = 0.5 \times ( {L}_{1} + {L}_{2} )$$

$$\sqrt{\frac{height ( cm ) \times weight ( kg )}{3600}} .$$

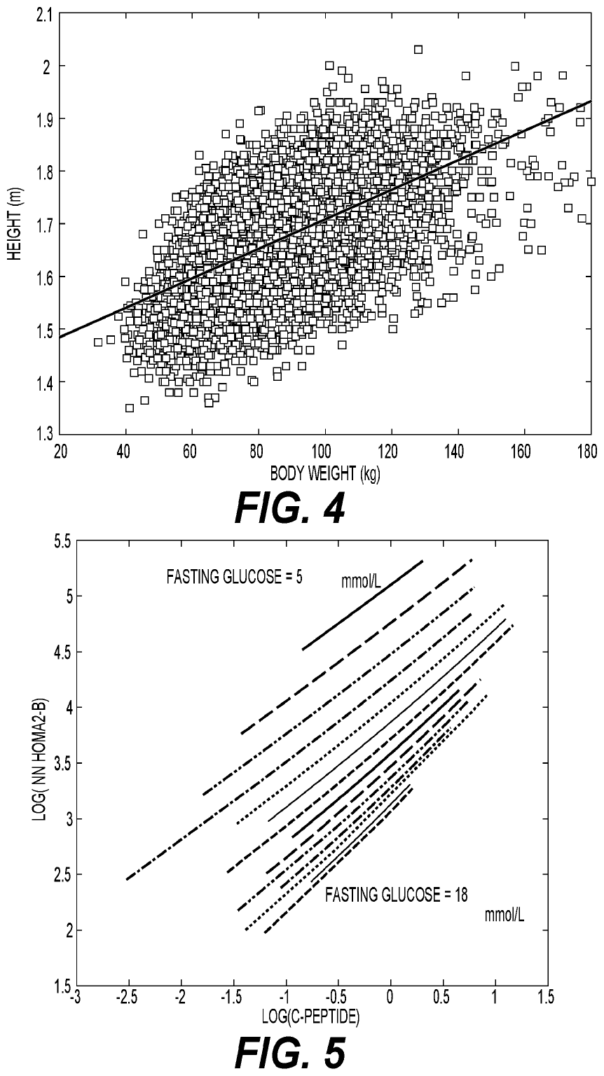

Information of height is obtained from linear regression of the training set, such as Height=0.0028×BW+1.4288, where height has the unit of m² and body (BW) of kg shown in FIG. 4. When creating virtual subjects from this regression, there is 10% of the nominal value for the standard deviation, and the range of height is set between [1, 2] m similar to that in the training set [1.35, 2.03] m.

### Modeling β-Cell Function and Insulin Resistance (HOMA2-B and HOMA2-IR)

Insulin resistance (IR) and B-cell function are clinically important diagnostic and assessment parameters in diabetes. In 1985 [11], the Homeostatic model assessment (HOMA) was developed to estimate the steady state β-cell function (HOMA-B) and insulin resistance (HOMA-IR) as percentages of a normal reference population values determined from hyper- and euglycaemic clamps and IVGTTs. The HOMA values are determined from basal (fasting) glucose uptake and fasting level of Insulin or C-peptide and, to a large extent, can differentiate β-cell deficiency from insulin resistance. However, these early estimates were not very reliable and were markedly influenced by uncertainty from the assays used to measure insulin and by the effects of stress and physical activity. HOMA was later expressed by a set of mathematical equations which was convenient for use in large-scale clinical studies. However, they still suffer from a variety of issues, like for example, the inability to account for variations in hepatic and peripheral glucose resistance, the contribution of circulating proinsulin. Most of the limitations of the earlier HOMAs were addressed in 1998, by the HOMA2 model [10], which recalibrated HOMA to give HOMA2-B and HOMA2-IR using currently available assays for insulin, specific insulin or C-peptide. This model also takes into account physiological processes that influence the concentrations of glucose and insulin used for the estimates of β-cell function and insulin sensitivity and has been validated against a variety of direct physiological measurements. In 2004, the HOMA2 calculator was made available to the community thereby providing clinicians and researchers with a simple and convenient tool to evaluate HOMA2-B and HOMA2-IR.

Ever since the HOMA2 calculator was made available it has been extensively used in the diabetes and metabolic research as well as in a variety of epidemiology studies. Given that HOMA2-B and HOMA2-IR characterize well the pathophysiology in individuals with abnormal glucose tolerance it is useful to have these models expressed in a convenient and accessible mathematical form as explicit functions of basal glucose, insulin or C-peptide measurements. Using the basal glucose and C-peptide measurements from a large diabetes population, closed-form expressions for HOMA2-B and HOMA2-IR which correctly reproduce the corresponding observed HOMA2-B and HOMA2-IR are provided.

### Development of a Mathematical Model for HOMA2-B

In order to develop a mathematical expression for HOMA2-B as a function of its dependent variables (fasting glucose and fasting C-peptide), we first explored how HOMA2-B varies as a function of one of the dependent variables while keeping the other variable constant. After observing some apparent nonlinearity between HOMA2-B and its dependent variables, we found that there is a linear log-log relationship between HOMA2-B and C-peptide at different fixed fasting glucose levels and used this relationship as a basis to develop a mathematical model for HOMA2-B. The linear relationship between log (HOMA2-B) and log(C-peptide) is then described by its slope and intercept which both depend on the (fixed) fasting glucose level. The dependence of the slope and intercept on fasting glucose is then determined to conclude the development of the HOMA2-B model.

We started the analysis by plotting (log-log) the HOMA2-B data against fasting C-peptide data at different fixed levels of fasting glucose. As shown in FIG. 5, at fixed low fasting glucose values, the dependence of ln(HOMA2-B) on ln(C-peptide) is almost linear. However, as the fasting glucose increases, the relationship gradually loses its linearity. To account for this behavior, the dependence of ln(HOMA2-B) on ln(C-peptide) is approximated by the expression (second order polynomial):

$$ln ( HOMA 2 ‐ B ) = A ( G ) \times {( ln ( {C}_{p} ) )}^{2} + B ( G ) \times ln ( {C}_{p} ) + C ( G ) ,$$

where A(G) accounts for the nonlinearity between HOMA2-B and C-peptide at higher levels of fasting glucose, B(G) describes the linear dependence, and C(G) is the intercept. Here the variables Cp and G are fasting C-peptide and fasting glucose, respectively. For the functions A(G), B(G) and C(G), we predefine a closed form expression determined their parameters by a least square fitting procedure from the training dataset:

$$A ( G ) = 0.0041 \times G - 0 \text{.0088}$$

$$B ( G ) = 0.0221 \times G + 0 \text{.5826}$$

$$C ( G ) = 59 \text{.1716} / ( G - 0.0178 \times {G}^{2} + 7.06 \; 51 ) .$$

Knowing these functions, the final expression for HOMA2-B can be rewritten as

$$HOMA 2 = exp ( A ( G ) \times {( ln ( {C}_{p} ) )}^{2} ) \times {C}_{p}^{B ( G )} \times exp ( C ( G ) )$$

Calculated Vs. Actual HOMA2-B on Training and Testing Data

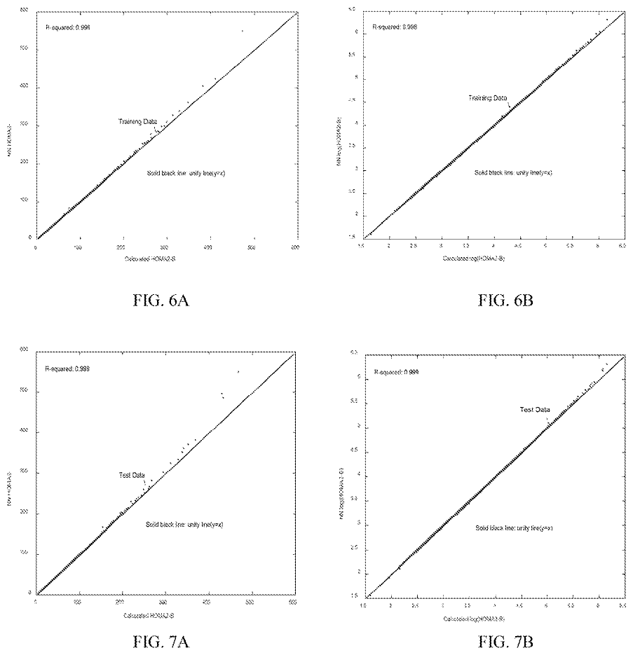

In FIGS. 6A-7B, the calculated HOMA2-B using the model above is plotted against the actual HOMA2-B both in normal and in log-log plot for the training data (in FIGS. 6A and 6B) and the test data (in FIGS. 7A and 7B). The excellent linear relationship indicates that the mathematical expression that estimates HOMA2-B from fasting glucose and fasting C-peptide describes accurately the actual HOMA2-B on the training data.

### Development of a Mathematical Model for HOMA2-IR

To develop a model for HOMA2-IR, we followed a strategy similar to the one used to develop the mathematical expression for HOMA2-B, but accounting for the fact that HOMA2-B and HOMA2-IR are markers of different physiological processes and their dependences on fasting glucose and C-peptide are likely to be analytically different. We explored the relationship between C-peptide and HOMA2-IR of the training dataset and found that at fixed glucose levels, HOMA2-IR depends linearly on C-peptide with zero intercept which depends on fasting glucose in a non-linear fashion determined from the training data. The detailed development is outlined below.

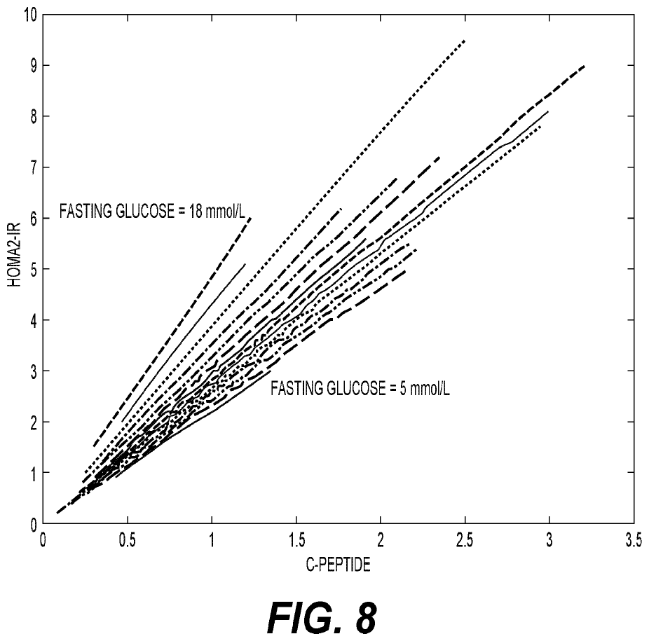

FIG. 8 illustrates the linear relationships between HOMA2-IR and C-peptide at different fixed fasting glucose levels.

Assuming a zero intercept and a slope varying with fasting glucose, a full mathematical expression for HOMA2-IR as a function of C-peptide and fasting glucose can be written as

$$HOMA 2 ‐ IR = {A}_{2} ( G ) \times {C}_{p} .$$

By varying the glucose levels, variations in the slope, A₂(G), are determined which increase nonlinearly with fasting glucose. We found that A₂(G) as function of G can be approximated well with a third order polynomial as follows:

$${A}_{2} ( G ) = 0.0022 \times {G}^{3} - 0.0607 \times {G}^{2} + 0.6321 \times G + 0.2588 ,$$

where the coefficients of the polynomial are determined by least square fitting using the training dataset.

Therefore, the mathematical expression for HOMA2-IR as a function of C-peptide and fasting glucose can be written as: HOMA2-IR=(0.0022×G³−0.0607×G²+0.6321×G+0.2588)×Cₚ.

Calculated Vs. Actual HOMA2-IR on Training and Testing Data

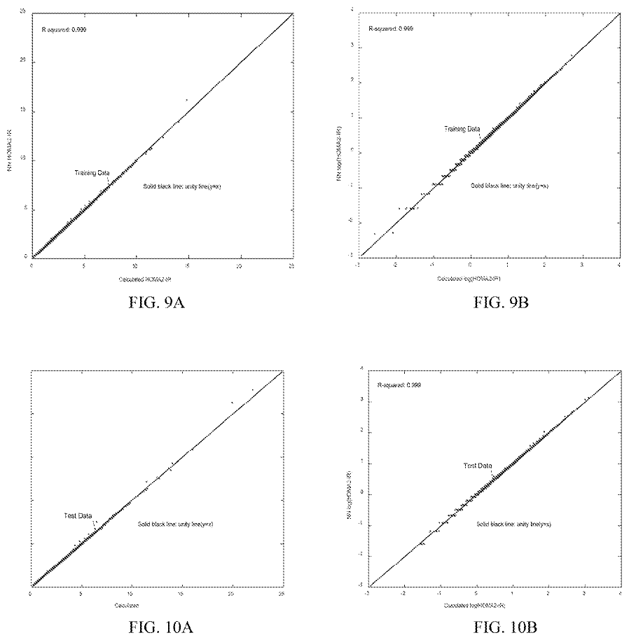

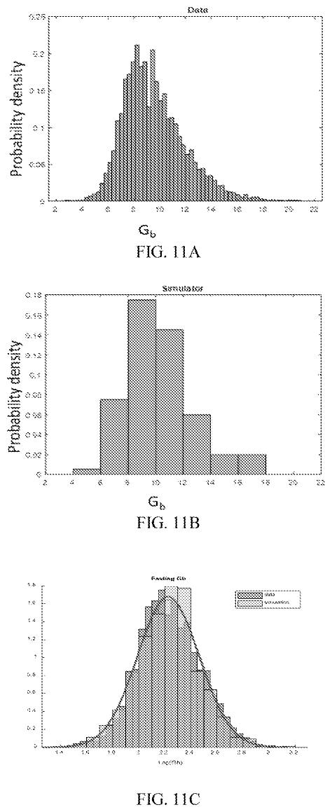

In FIGS. 9A-10B, the calculated HOMA2-B using the model above is plotted against the actual HOMA2-B both in normal and in log-log plot for the training data (in FIGS. 9A and 9B) and the test data (in FIGS. 10A and 10B). The figures show that, on the training data, the closed-form mathematical expression accurately describes the actual data with an R-squared value close to 1.0.

### Matching the Overall, Population-Level, In Vivo Vs in Silico Physiological Distributions

Herein, the method achieves creation of a virtual population with distributions of physiological and demographic variables that are similar to those in the training data set. The parameter values of virtual subjects are generated from a vector of mean values, and a covariance matrix of these parameters. The mean and covariance values of corresponding variables can be adjusted to create simulated outcomes to match the physiological variables of fasting glucose, fasting C-peptide, HOMA2-B, HOMA2-IR, and hemoglobin A1c (HbA1c) in the training set.

### Fasting Glucose

In the Type 2 Diabetes (T2D) model, the variable of fasting glucose (G_{b}) may be the corresponding one of that in the data. The first step is to enforce that G_{b} in the model has a matched distribution from the training data set, such as the values of mean and variance. The distributions of fasting glucose in real and simulated data are shown in FIGS. 11A and 11B, respectively. Since the variable of G_{b} is log-normally distributed in the parameters space, these values are based on the log-normal of fasting glucose. The adjustment of G_{b} is given as

$$mean ( ln ( {G}_{b} ) ) = 1.005 \times mean ( ln ( {G}_{b \_ training} ) )$$

$$ratio = \frac{var ( ln ( {G}_{b \_ training} ) )}{var ( ln ( {G}_{b \_ current} ) )} .$$

The row and the column of G_{b} in the covariance matrix is multiplied with the value of \sqrt{ratio} to have the same variance of G_{b} as that in the training set. The outcome is shown in FIG. 11C.

### Fasting C-Peptide, HOMA2-B, and HOMA2-IR

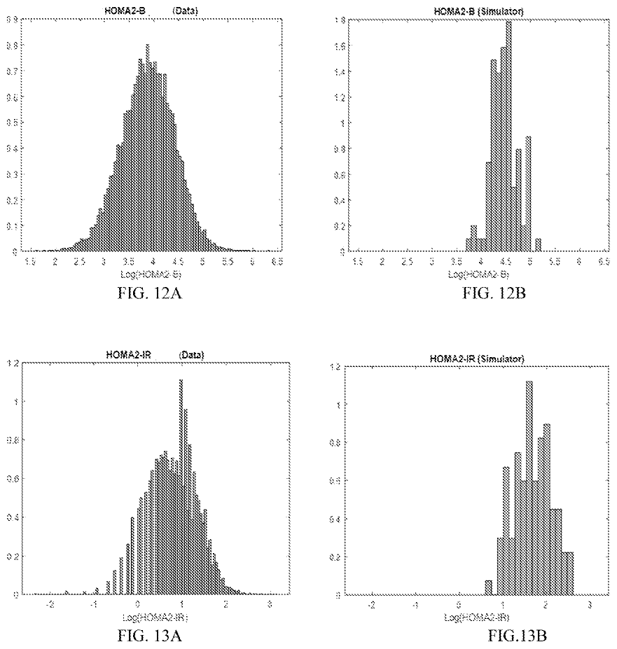

The original and simulated results of HOMA2-B and HOMA2-IR are respectively shown in FIGS. 12A-12B and FIGS. 13A-13B, and provide for a smaller variance when compared to the data. Since their values are determined by fasting glucose and fasting C-peptide, the basal insulin secretion rate (SR_{b}), which is associated with fasting C-peptide, will be tuned to match the fasting C-peptide, HOMA2-B and HOMA2-IR. There are two aspects of SR_{b} to be tuned: mean and the variance such as

$$mean ( {SR}_{b} ) = {r}_{1} \times \frac{{G}_{b}}{100}$$

$$var ( {SR}_{b} ) = {r}_{2} \times var ( {SR}_{b \_ current} ) .$$

The mean value of SR_{b} is adapted from to correlate with fasting glucose.

The cost function (f) of searching for optimal r₁ and r₂ is the least-squares of empirical cumulative distribution function (ECDF) difference of ln(HOMA2-B) and ln(HOMA2-IR) between the values from training set and simulation results:

$$min f = min \{ \sum {( ECDF ( ln ( HOMA 2 ‐ B\_training ) ) - ECDF ( ln ( HOMA 2 ‐ B\_sim ) ) )}^{2} + \sum {( ECDF ( ln ( HOMA 2 ‐ IR\_training ) ) - ECDF ( ln ( HOMA 2 ‐ IR\_sim ) ) )}^{2} \} .$$

### Fine-Tuning of Model Parameters

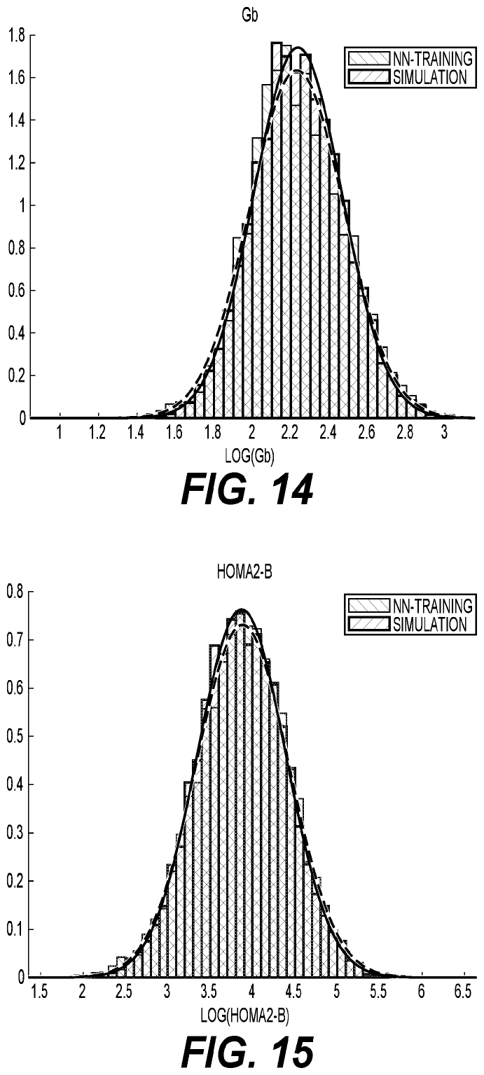

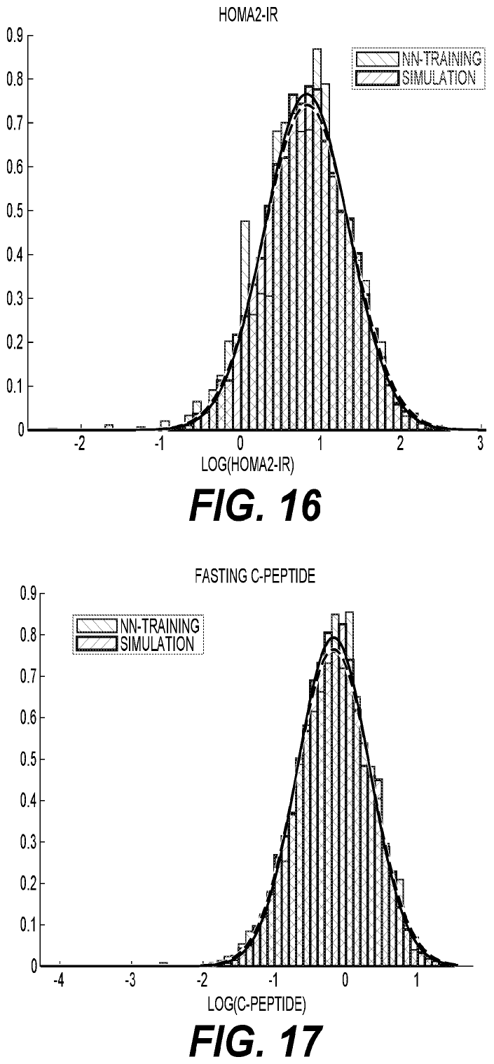

The results of tuning SR_{b} are: r₁=1.53, r₂=33.6. The distributions of variables fasting glucose (Gb), HOMA2-B, HOMA2-IR, and fasting C-peptide are shown from FIGS. 14 to 17.

### Matching of Clinical Subtypes: Results by Clusters

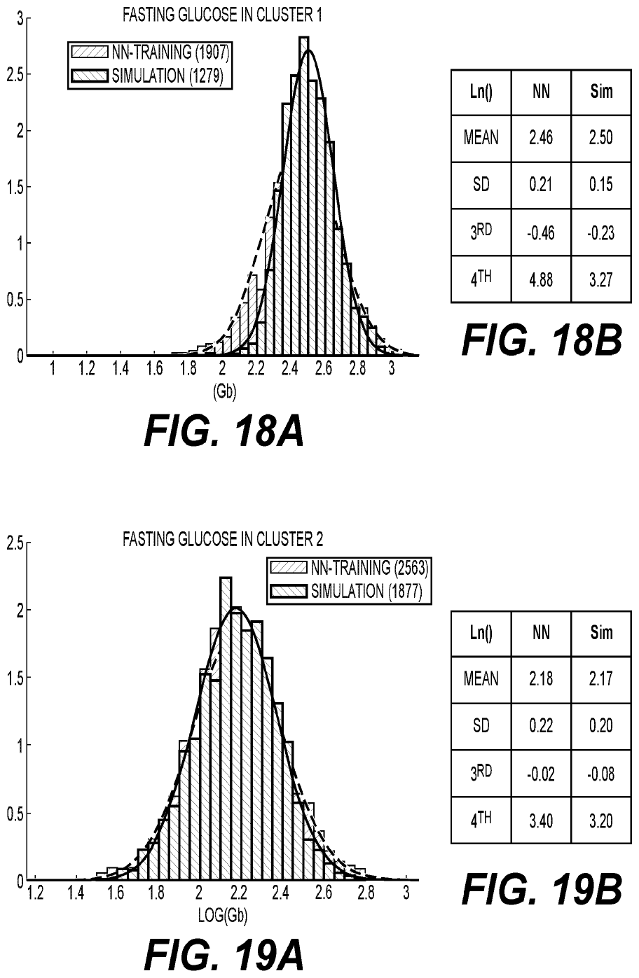

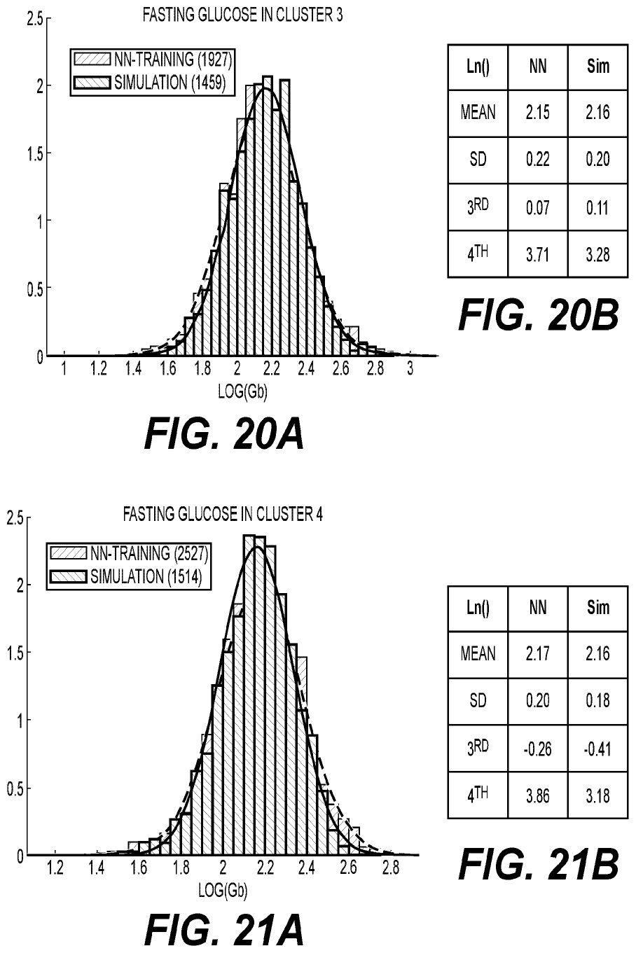

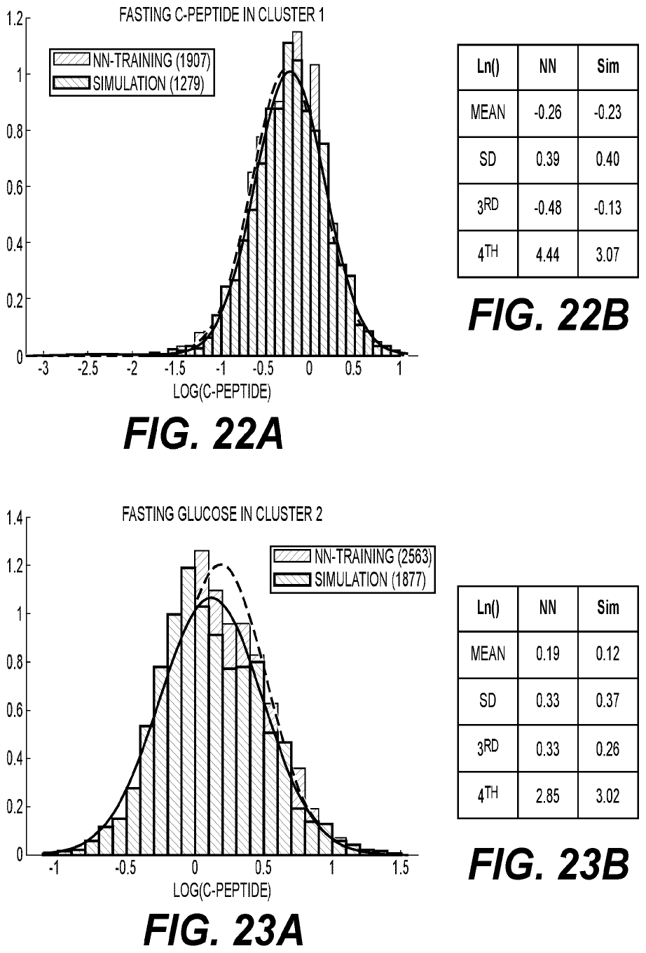

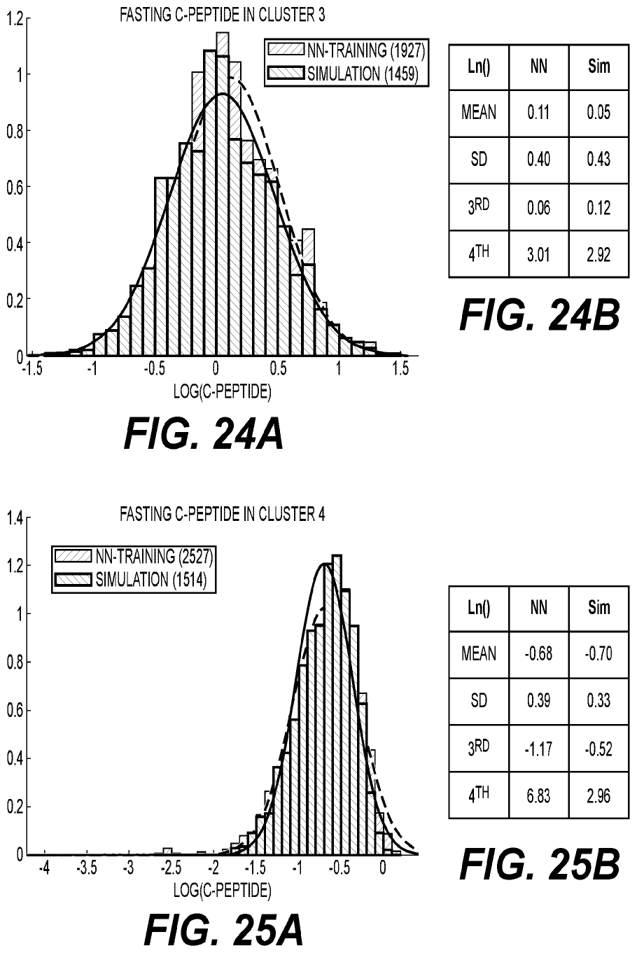

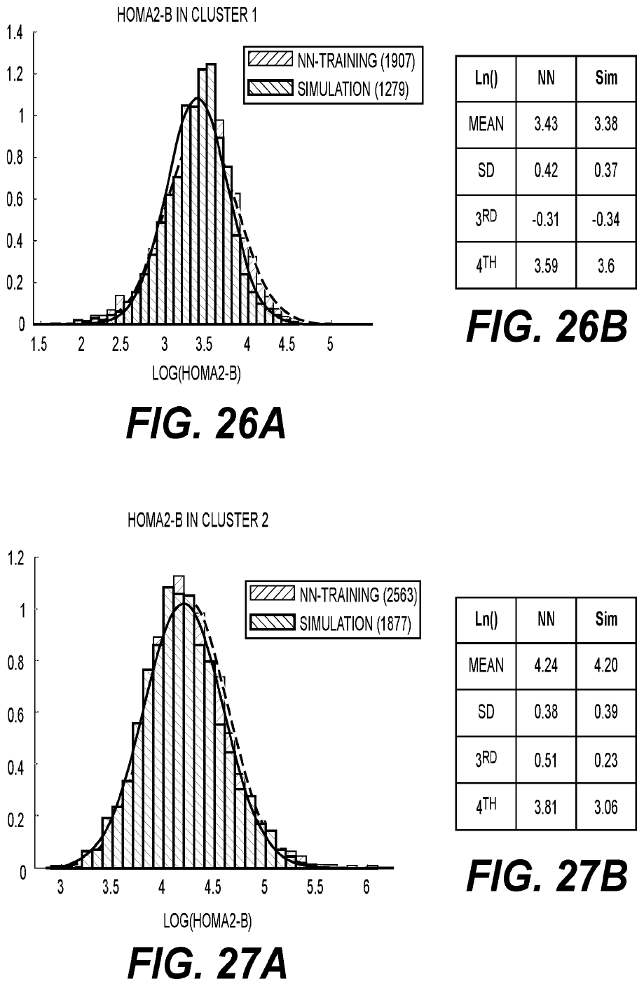

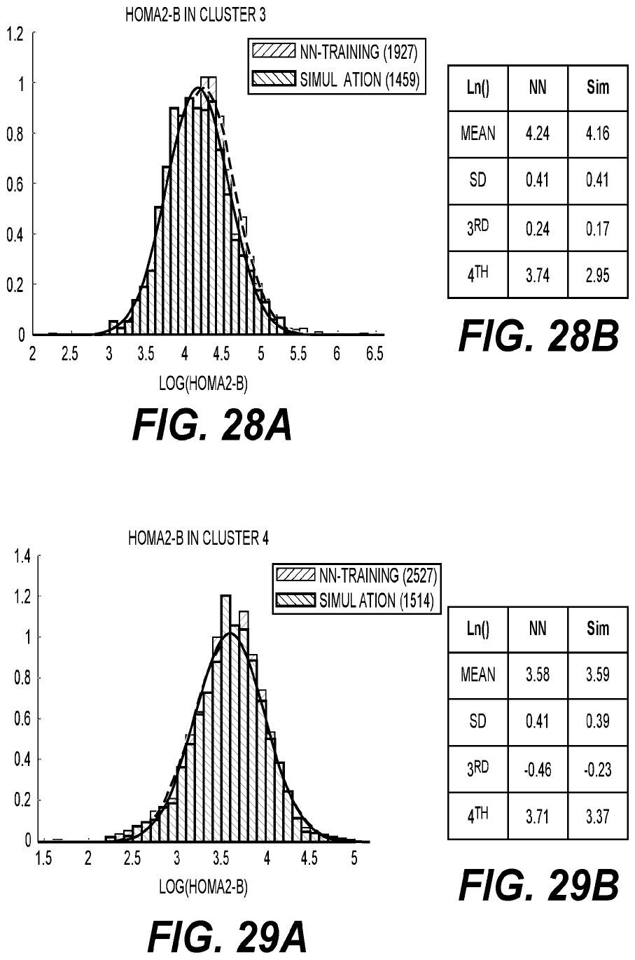

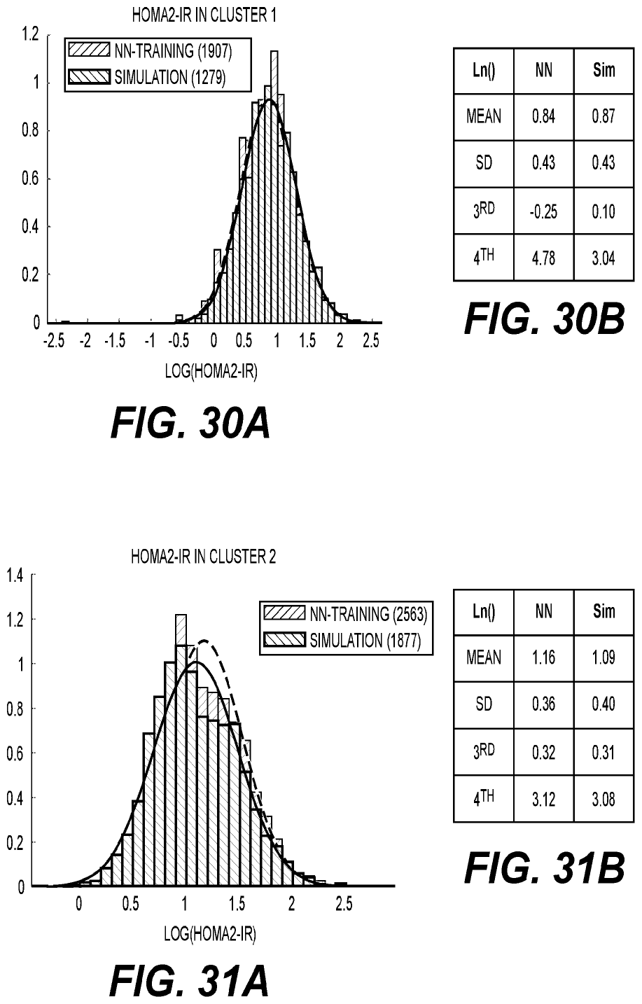

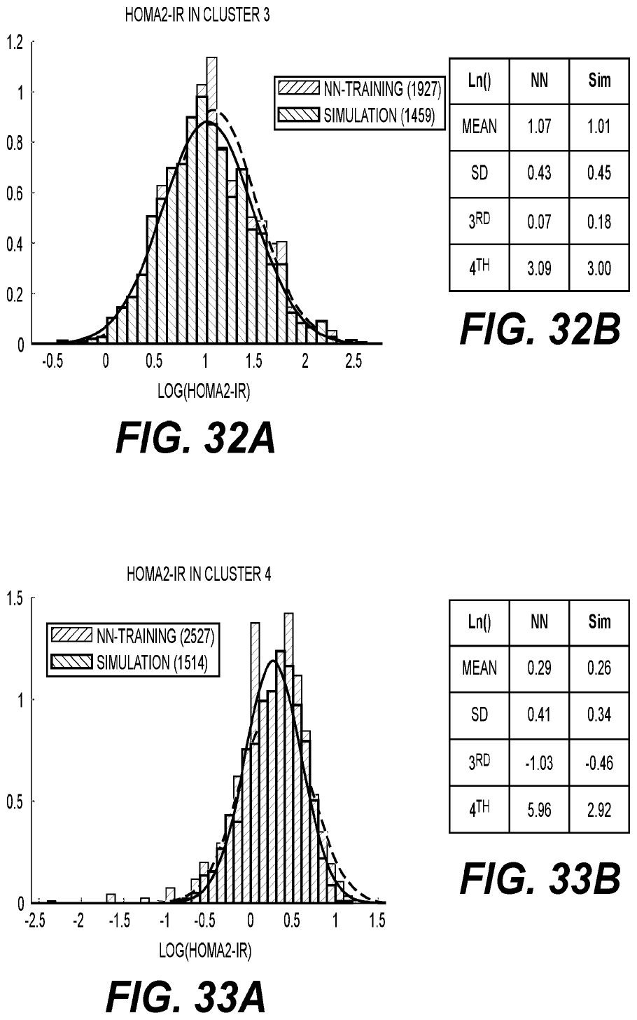

The initial simulation results are checked by clusters from FIGS. 18A to 33B. In the associated tables, the values of mean, standard deviation (SD), skewness (3^{rd}), and kurtosis (4^{th}) are demonstrated. Ln represents taking the natural logarithm of the values. The clusters of 1 through 4 represent the clusters of 2 through 5 respectively in [15]. In these regards, the shown distributions in conjunction with the aforementioned measures can each provide for absolute differences representing predetermined proximities as between training data and simulation results. The overall variables exhibit significant match, except the left tail of fasting glucose in cluster 1, and shifts of BMI and BW in cluster 1 and 4. The mismatch of fasting glucose can be due to dispensation of possible treatments, which are not addressed in the data. As for the mismatch of BMI and BW, the same can be addressed by adjusting the covariance matrix of virtual subjects.

Method Validation and Prediction of Clinical Subtypes in the Test Data Set Classification into Clinical Subtypes (Clusters)

After fixing all model parameters, and the mapping procedure relative to the Training Data, the method was applied to the Test data set and judged for its ability to predict accurately clinical subtypes/cluster membership (see Table 1 below).

TABLE 1

| In silico data (6156) | | | NN Test data (8922) | |
|---|---|---|---|---|
| Cluster | Number | % of the total | number | % of the total |
| 1 | 1311 | 21.30 | 1907 | 21.37 |
| 2 | 1719 | 27.92 | 2563 | 28.73 |
| 3 | 1283 | 20.84 | 1926 | 21.59 |
| 4 | 1843 | 29.94 | 2526 | 28.31 |

### Correlation Structure

The correlation matrix relative to the test set and simulated subjects is demonstrated in Tables 2 and 3 below.

TABLE 2

| | BW | Gb | C-pep | HOMA2-B | HOMA2-IR |
|---|---|---|---|---|---|
| BW | 1 | 0.0854 | 0.4664 | 0.2629 | 0.4456 |
| Gb | | 1 | 0.0928 | −0.5758 | 0.3201 |
| C-pep | | | 1 | 0.6062 | 0.9489 |
| HOMA2-B | | | | 1 | 0.4253 |
| HOMA2-IR | | | | | 1 |

TABLE 3

| | BW | Gb | C-pep | HOMA2-B | HOMA2-IR |
|---|---|---|---|---|---|
| BW | 1 | 0.1208 | 0.3920 | 0.2042 | 0.4080 |
| Gb | | 1 | −0.0036 | −0.5858 | 0.1616 |
| C-pep | | | 1 | 0.7232 | 0.9799 |
| HOMA2-B | | | | 1 | 0.6051 |
| HOMA2-IR | | | | | 1 |

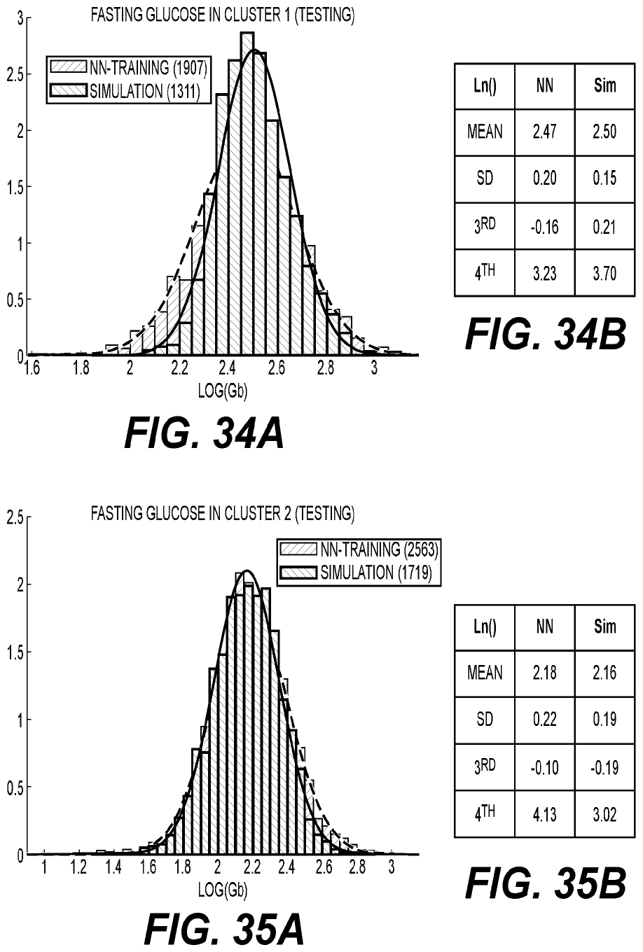

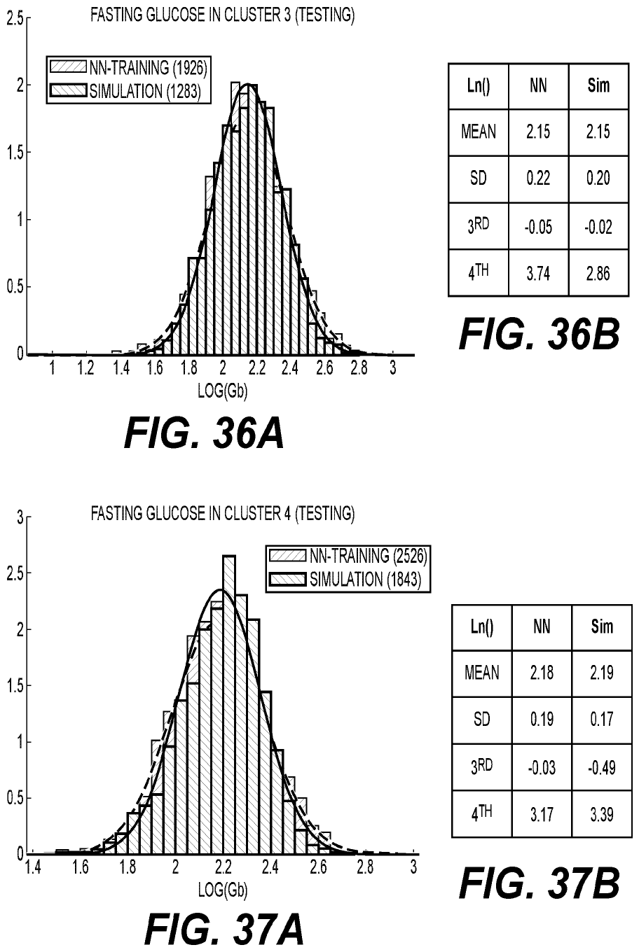

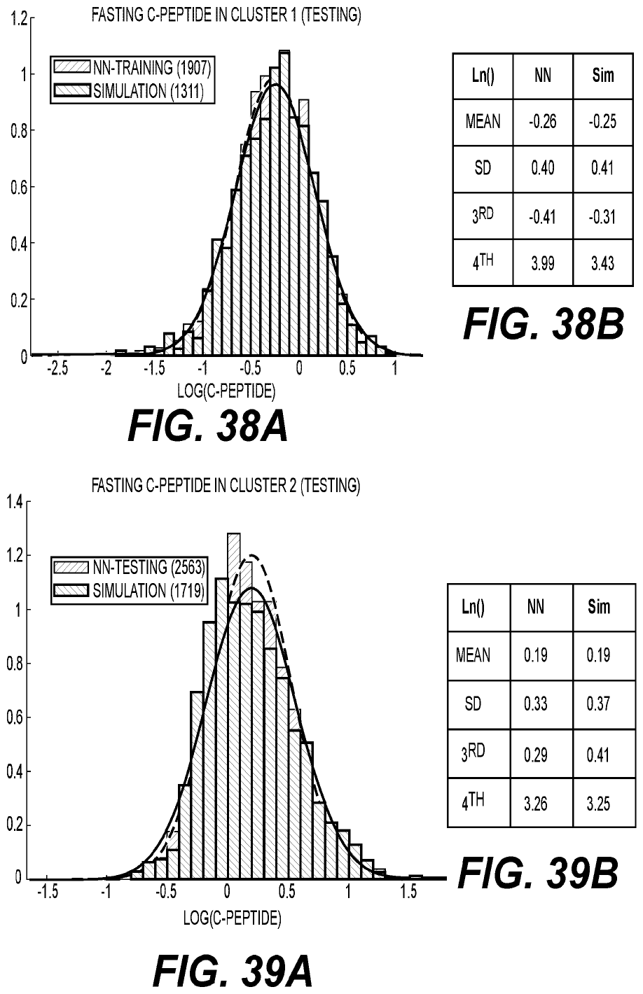

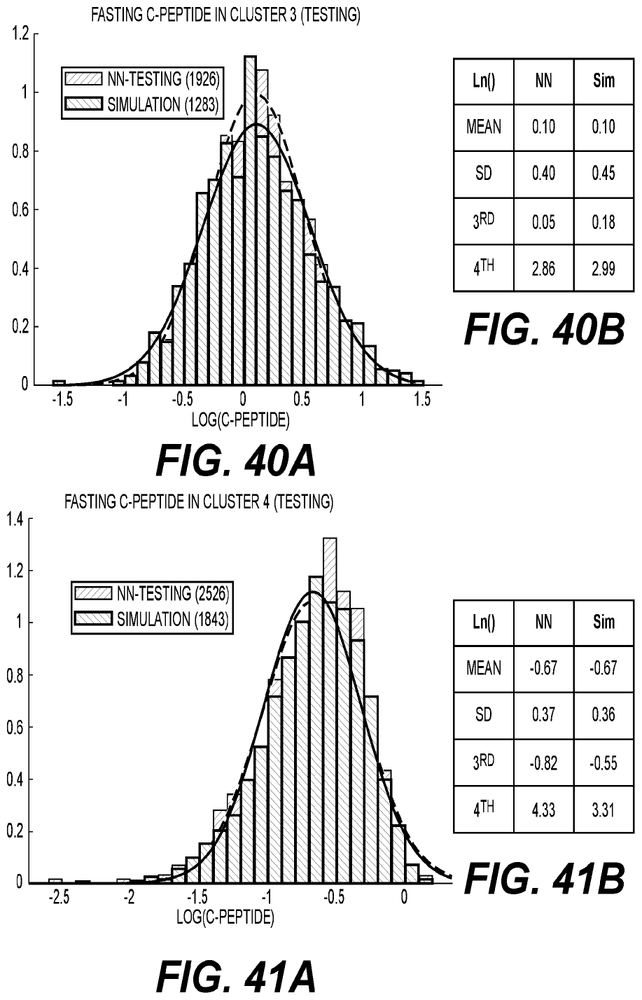

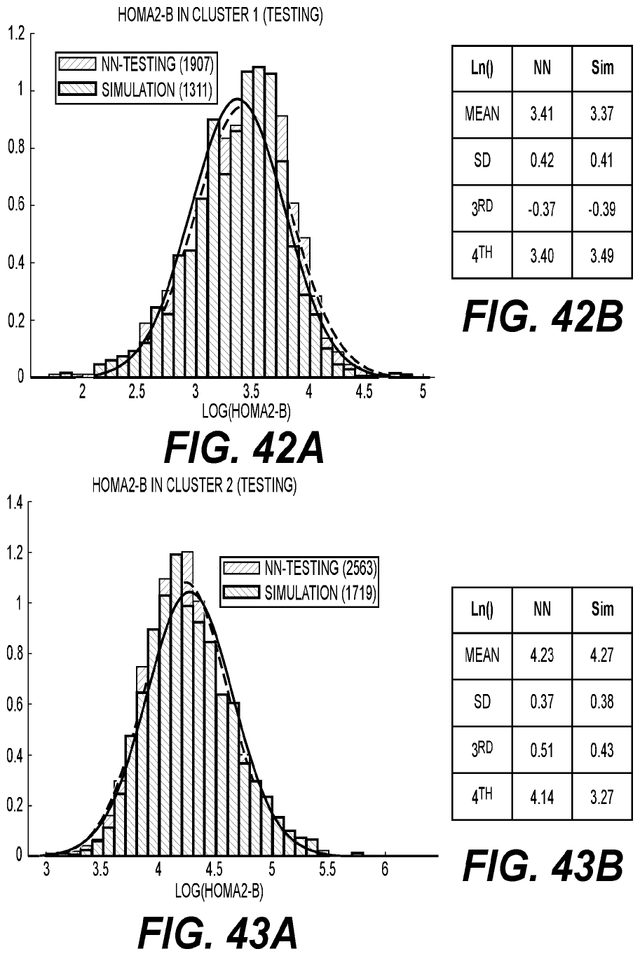

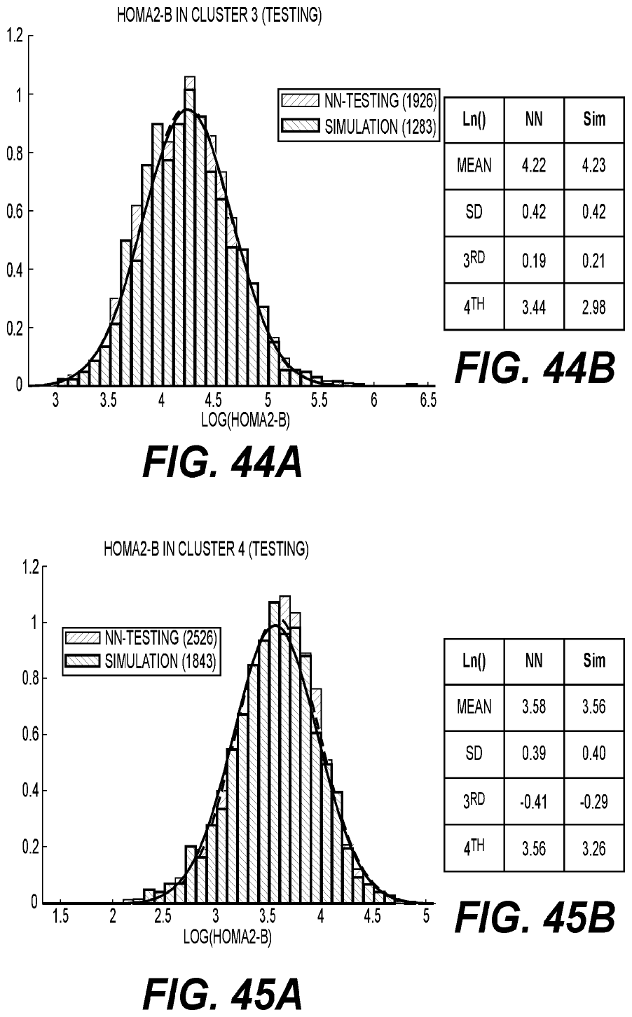

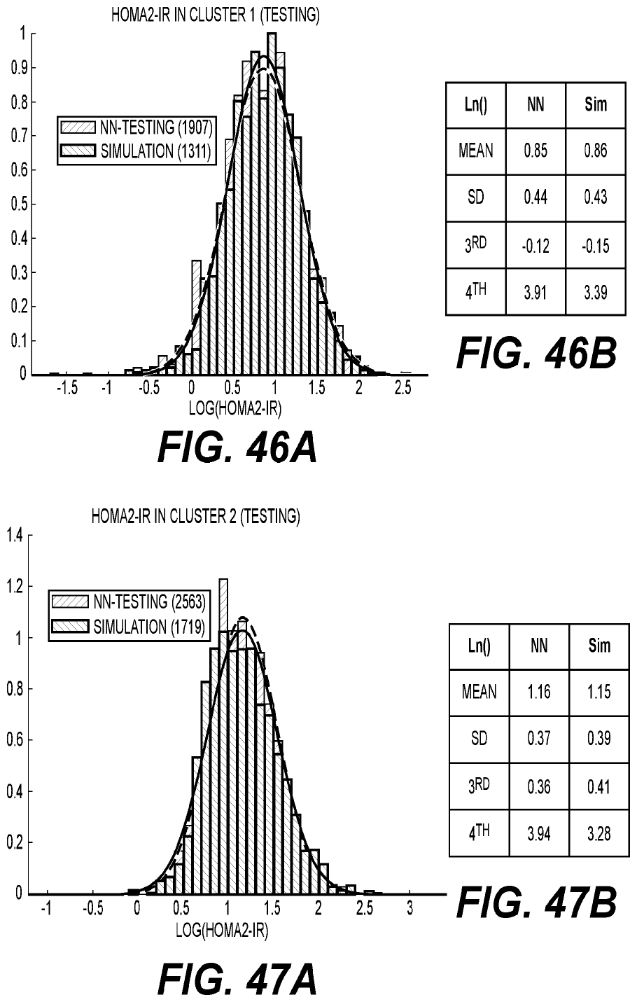

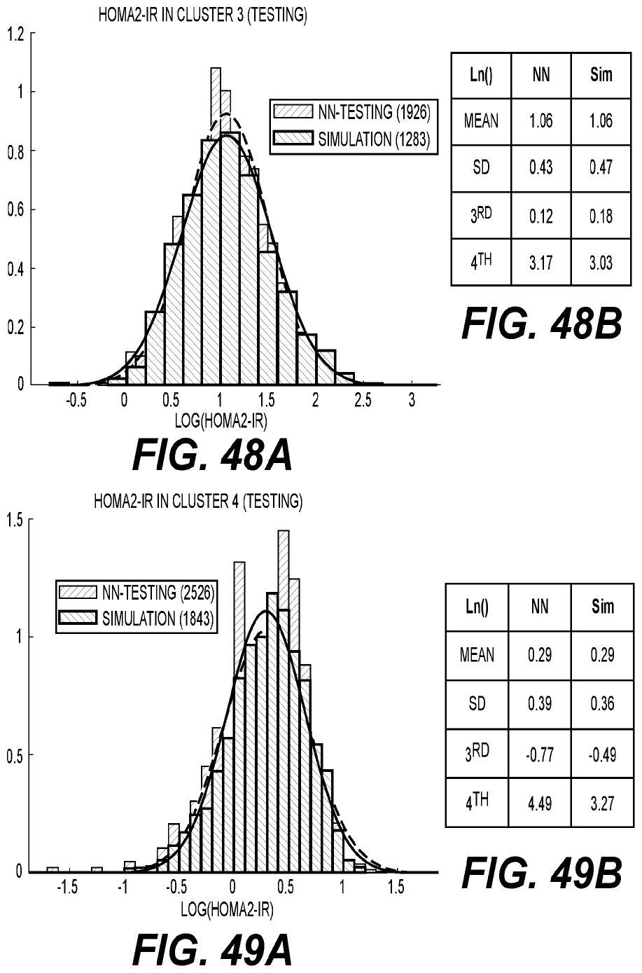

Similar to comparison of operation of the method as against the training data with respect to at least the physiological variables of fasting glucose, HOMA2-B, HOMA2-IR, and fasting C-peptide (as shown in FIGS. 18A-33B), validation of the method herein further encompasses performing similar comparisons with respect to these variables as against the testing data, as is shown in FIGS. 34A-49B. Accordingly, one or more aspects of the validation of the method may include (a) substantially maintaining cluster memberships (see Table 1 above), (b) substantially maintaining exemplary correlation(s) similar to those shown in the examples of above Tables 2 and 3, as well as (c) obtaining the aforementioned testing data comparisons showing substantially similar results to those shown by the exemplary, substantially matching distributions of FIGS. 34A-49B as between the testing data and the data generated by the method. In the associated tables, the values of mean, standard deviation (SD), skewness (3rd), and kurtosis (4th) are demonstrated. Ln represents taking the natural logarithm of the values. The clusters of 1 through 4 represent the clusters of 2 through 5 respectively in [15]. In these regards, the shown distributions in conjunction with the aforementioned measures can each provide for absolute differences representing predetermined proximities as between (a) training or testing data and (b) simulation results.

Thus, in view of the above, one of skill in the art will appreciate that there is provided a method for quantitative physiological assessment and prediction of clinical subtypes of glucose metabolism disorders, including but not limited to, Type 1 diabetes, obesity, pre-diabetes, gestational diabetes, or variants of Type 2 diabetes discussed herein. The method allows a virtual population of in silico entities to be created, reproducing faithfully the clinical subtype distributions observed in vivo. Potential applications of this method may include, but are not limited to: (a) enabling in silico experiments assessing and/or predicting, for a real patient, treatment or intervention outcomes for a given population/clinical-subtype level; (b) pre-clinical testing of properties of new medications; (c) augmenting limited clinical trial data with in silico experiments to an extent that the domain of their validity and test corner cases may not be observed in vivo.

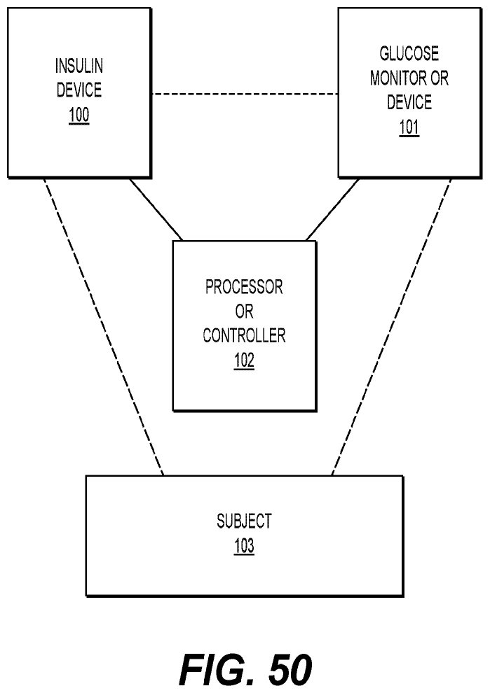

With respect to FIGS. 50-55, and when referring to FIG. 50, there is shown a high level functional block diagram of an artificial pancreas (AP) by which one or more aspects of the discussed method may be coordinated according to embodiments herein.

As shown, a processor or controller 102, may be configured to implement each of the prediction module and insulin infusion control module discussed above and to communicate with a CGM 101, and optionally with an insulin device 100 enabled to deliver insulin. The glucose monitor or device 101 may communicate with a subject 103 to monitor glucose levels thereof. The processor or controller 102 may be configured to include all necessary hardware and/or software necessary to perform the required instructions to achieve the aforementioned tasks. Optionally, the insulin device 100 may communicate with the subject 103 to deliver insulin thereto. The glucose monitor 101 and the insulin device 100 may be implemented as separate devices or as a single device in combination. The processor 102 may be implemented locally in the glucose monitor 101, the insulin device 100, or as a standalone device (or in any combination of two or more of the glucose monitor, insulin device, or a standalone device). The processor 102 or a portion of the AP may be located remotely, such that the AP may be operated as a telemedicine device.

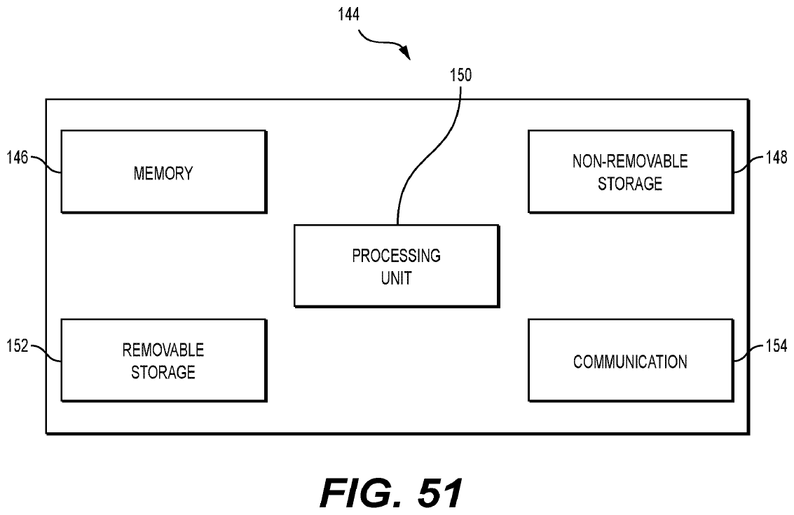

Referring to FIG. 51, a computing device 144 may implement the AP and may typically include at least one processing unit 150 and memory 146. Depending on the exact configuration and type of computing device, memory 146 may be volatile (such as RAM), non-volatile (such as ROM, flash memory, etc.) or some combination of the two.

Additionally, computing device 144 may also have other features and/or functionality. For example, the device could also include additional removable and/or non-removable storage including, but not limited to, magnetic or optical disks or tape, as well as writable electrical storage media. Such additional storage may be represented as removable storage 152 and non-removable storage 148. Computer storage media may include volatile and nonvolatile, removable and non-removable media implemented in any method or technology for storage of information such as computer readable instructions, data structures, program modules or other data. The memory, the removable storage and the non-removable storage may comprise examples of computer storage media. Computer storage media may include, but not be limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, one or more components of the AP.

The computer device 144 may also contain one or more communications connections 154 that allow the device to communicate with other devices (e.g. other computing devices). The communications connections may carry information in a communication media. Communication media may typically embody computer readable instructions, data structures, program modules or other data in a modulated data signal, such as a carrier wave or other transport mechanism and includes any information delivery media. The term “modulated data signal” may include a signal that has one or more of its characteristics set or changed in such a manner as to encode, execute, or process information in the signal. By way of example, and not limitation, communication medium may include wired media such as a wired network or direct-wired connection, and wireless media such as radio, RF, infrared and other wireless media. As discussed above, the term computer readable media as used herein may include both storage media and communication media.

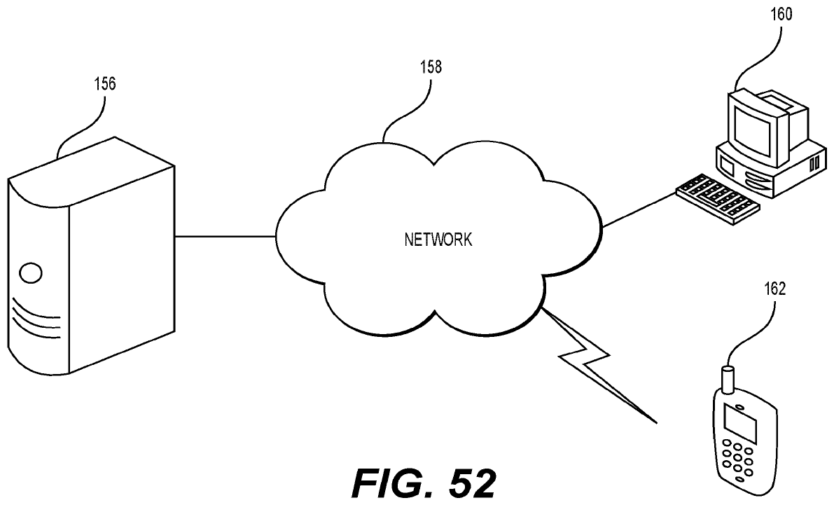

In addition to a stand-alone computing machine, embodiments herein may also be implemented on a network system comprising a plurality of computing devices that may in communication via a network, such as a network with an infrastructure or an ad hoc network. The network connection may include wired connections or wireless connections. For example, FIG. 52 illustrates a network system in which embodiments herein may be implemented. In this example, the network system may comprise a computer 156 (e.g., a network server), network connection means 158 (e.g., wired and/or wireless connections), a computer terminal 160, and a PDA (e.g., a smartphone) 162 (or other handheld or portable device, such as a cell phone, laptop computer, tablet computer, GPS receiver, mp3 player, handheld video player, pocket projector, etc. or other handheld devices (or non-portable devices) with combinations of such features). In an embodiment, it should be appreciated that the module listed as 156 may implement a CGM. In an embodiment, it should be appreciated that the module listed as 156 may be a glucose monitor device, an artificial pancreas, and/or an insulin device. Any of the components shown or discussed with FIG. 52 may be multiple in number. Embodiments herein may be implemented in anyone of the aforementioned devices. For example, execution of the instructions or other desired processing may be performed on the same computing device that is anyone of 156, 160, and 162. Alternatively, an embodiment may be performed on different computing devices of the network system. For example, certain desired or required processing or execution may be performed on one of the computing devices of the network (e.g. server 156 and/or a CGM), whereas other processing and execution of the instruction can be performed at another computing device (e.g., terminal 160) of the network system, or vice versa. In fact, certain processing or execution may be performed at one computing device (e.g. server 156 and/or insulin device, artificial pancreas, or CGM); and the other processing or execution of the instructions may be performed at different computing devices that may or may not be networked. For example, such certain processing may be performed at terminal 160, while the other processing or instructions may be passed to device 162 where the instructions may be executed. This scenario may be of particular value especially when the PDA 162 device, for example, accesses the network through computer terminal 160 (or an access point in an ad hoc network). For another example, software comprising the instructions may be executed, encoded or processed according to one or more embodiments herein. The processed, encoded or executed instructions may then be distributed to customers in the form of a storage media (e.g. disk) or electronic copy.

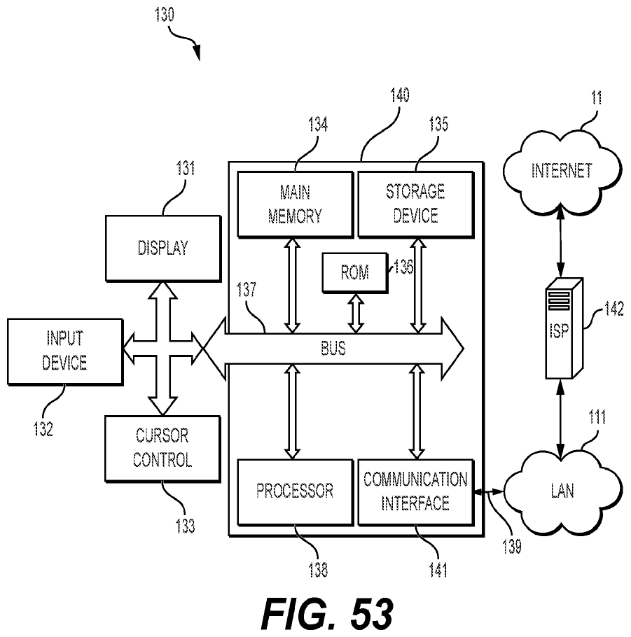

FIG. 53 illustrates a block diagram that of a system 130 including a computer system 140 and the associated Internet 11 connection upon which an embodiment may be implemented. Such configuration may typically used for computers (i.e., hosts) connected to the Internet 11 and executing software on a server or a client (or a combination thereof). A source computer such as laptop, an ultimate destination computer and relay servers, for example, as well as any computer or processor described herein, may use the computer system configuration and the Internet connection shown in FIG. 53. The system 140 may take the form of a portable electronic device such as a notebook/laptop computer, a media player (e.g., a MP3 based or video player), a cellular phone, a Personal Digital Assistant (PDA), a CGM, an AP, an insulin delivery device, an image processing device (e.g., a digital camera or video recorder), and/or any other handheld computing devices, or a combination of any of these devices. Note that while FIG. 53 illustrates various components of a computer system, it is not intended to represent any particular architecture or manner of interconnecting the components; as such, details of such interconnection are omitted. It will also be appreciated that network computers, handheld computers, cell phones and other data processing systems which have fewer components or perhaps more components may also be used. The computer system of FIG. 53 may, for example, be an Apple Macintosh computer or Power Book, or an IBM compatible PC. Computer system 140 may include a bus 137, an interconnect, or other communication mechanism for communicating information, and a processor 138, commonly in the form of an integrated circuit, coupled with bus 137 for processing information and for executing the computer executable instructions. Computer system 140 may also include a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 may further include a Read Only Memory (ROM) 136 (or other non-volatile memory) or other static storage device coupled to bus 137 for storing static information and instructions for processing by processor 138. A storage device 135, such as a magnetic disk or optical disk, a hard disk drive for reading from and writing to a hard disk, a magnetic disk drive for reading from and writing to a magnetic disk, and/or an optical disk drive (such as a DVD) for reading from and writing to a removable optical disk, may be coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer readable media may provide non-volatile storage of computer readable instructions, data structures, program modules and other data for the general purpose computing devices. Typically, computer system 140 may include an Operating System (OS) stored in a non-volatile storage for managing the computer resources and may provide the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of OSs may include Microsoft Windows, Mac OS X, and Linux.

The term “processor” may include any integrated circuit or other electronic device (or collection of such electronic devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., a silicon “die”), or may be distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display may allow a user to view, enter, and/or edit information that may be relevant to the operation of the system. An input device 132, including alphanumeric and other keys, may be coupled to bus 137 for communicating information and command selections to processor 138. Another type of user input device may include cursor control 133, such as a mouse, a trackball, or cursor direction keys for communicating direction information and command selections to processor 138, and for controlling cursor movement on display 131. Such an input device may include two degrees of freedom in two axes, a first axis (e.g., x) and a second axis (e.g., y), that may allow the device to specify positions in a plane.

The computer system 140 may be used for implementing the methods and techniques described herein. According to an embodiment, those methods and techniques may be performed by computer system 140 in response to processor 138 executing one or more sequences of one or more instructions contained in main memory 134. Such instructions may be read into main memory 134 from another computer readable medium, such as storage device 135. Execution of the sequences of instructions contained in main memory 134 may cause processor 138 to perform the process steps described herein. In alternative embodiments, hardwired circuitry may be used in place of or in combination with software instructions to implement the arrangement. Thus, embodiments of the invention may not be limited to any specific combination of hardware circuitry and software.

The term “computer readable medium” (or “machine readable medium”) as used herein is an extensible term that refers to any medium or any memory, that participates in providing instructions to a processor, (such as processor 138), for execution, or any mechanism for storing or transmitting information in a form readable by a machine (e.g., a computer). Such a medium may store computer-executable instructions to be executed by a processing element and/or control logic, and data which may be manipulated by a processing element and/or control logic, and may take many forms, including but not limited to, non-volatile medium, volatile medium, and transmission medium. Transmission media may include coaxial cables, copper wire and fiber optics, including the wires that comprise bus 137. Transmission media may also take the form of acoustic or light waves, such as those generated during radio-wave and infrared data communications, or other form of propagated signals (e.g., carrier waves, infrared signals, digital signals, etc.). Common forms of computer readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 138 for execution. For example, the instructions may initially be carried on a magnetic disk of a remote computer. The remote computer may load the instructions into its dynamic memory and send the instructions over a telephone line using a modem. A modem local to computer system 140 may receive the data on the telephone line and use an infra-red transmitter to convert the data to an infra-red signal. An infra-red detector may receive the data carried in the infra-red signal, and appropriate circuitry may place the data on bus 137. Bus 137 may carry the data to main memory 134, from which processor 138 may retrieve and execute the instructions. The instructions received by main memory 134 may optionally be stored on storage device 135 either before or after execution by processor 138.

Computer system 140 may also include a communication interface 141 coupled to bus 137. Communication interface 141 may provide a two-way data communication coupling to a network link 139 that may be connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 may typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 may send and receive electrical, electromagnetic or optical signals that may carry digital data streams representing various types of information.

Network link 139 may typically provide data communication through one or more networks to other data devices. For example, network link 139 may provide a connection through local network 111 to a host computer or to data equipment operated by an Internet Service Provider (ISP) 142. ISP 142, in turn, may provide data communication services through the world wide packet data communication network Internet 11. Local network 111 and Internet 11 may both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

In view of the above, minimization and/or prevention of the occurrence of hypoglycemia through use of the AP discussed herein may be readily applicable into devices with (for example) limited processing power, such as glucose, insulin, and AP devices, and may be implemented and utilized with the related processors, networks, computer systems, internet, and components and functions according to the schemes disclosed herein.

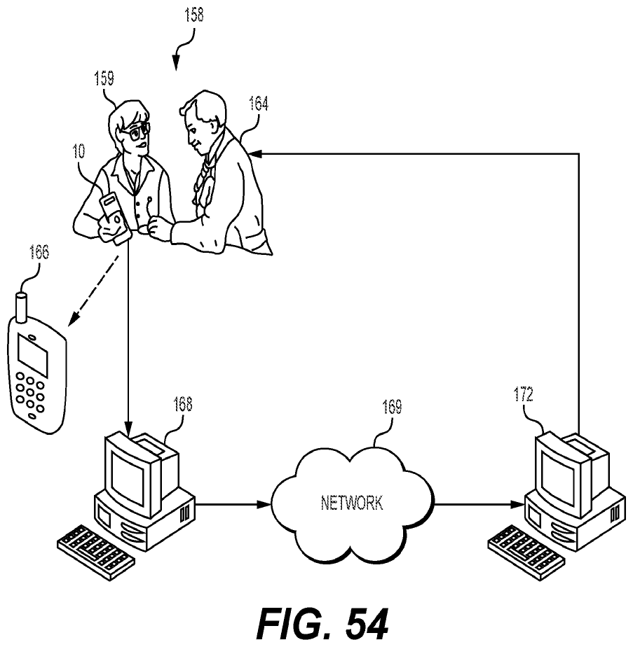

Referring to FIG. 54, there is shown an exemplary system in which examples of the invention may be implemented. In an embodiment, the CGM, the AP or the insulin device may be implemented by a subject (or patient) locally at home or at another desired location. However, in an alternative embodiment, one or more of the above may be implemented in a clinical setting. For instance, referring to FIG. 54, a clinical setup 158 may provide a place for doctors (e.g., 164) or clinician/assistant to diagnose patients (e.g., 159) with diseases related with glucose, and related diseases and conditions. A CGM 10 may be used to monitor and/or test the glucose levels of the patient—as a standalone device. It should be appreciated that while only one CGM 10 is shown in the figure, the system may include other AP components. The system or component, such as the CGM 10, may be affixed to the patient or in communication with the patient as desired or required. For example, the system or combination of components thereof—including a CGM 10 (or other related devices or systems such as a controller, and/or an AP, an insulin pump, or any other desired or required devices or components)—may be in contact, communication or affixed to the patient through tape or tubing (or other medical instruments or components) or may be in communication through wired or wireless connections. Such monitoring and/or testing may be short term (e.g., a clinical visit) or long term (e.g., a clinical stay). The CGM may output results that may be used by the doctor (clinician or assistant) for appropriate actions, such as insulin injection or food feeding for the patient, or other appropriate actions or modeling. Alternatively, the CGM 10 may output results that may be delivered to computer terminal 168 for instant or future analyses. The delivery may be through cable or wireless or any other suitable medium. The CGM 10 output from the patient may also be delivered to a portable device, such as PDA 166. The CGM 10 output may also be delivered to a glucose monitoring center 172 for processing and/or analyzing. Such delivery can be accomplished in many ways, such as network connection 170, which may be wired or wireless.

In addition to the CGM 10 output, errors, parameters for accuracy improvements, and any accuracy related information may be delivered, such as to computer 168, and/or glucose monitoring center 172 for performing error analyses. Doing so may provide centralized monitoring of accuracy, modeling and/or accuracy enhancement for glucose centers, relative to assuring a reliable dependence upon glucose sensors.

Examples of the invention may also be implemented in a standalone computing device associated with the target glucose monitoring device. An exemplary computing device (or portions thereof) in which examples of the invention may be implemented is schematically illustrated in FIG. 51.

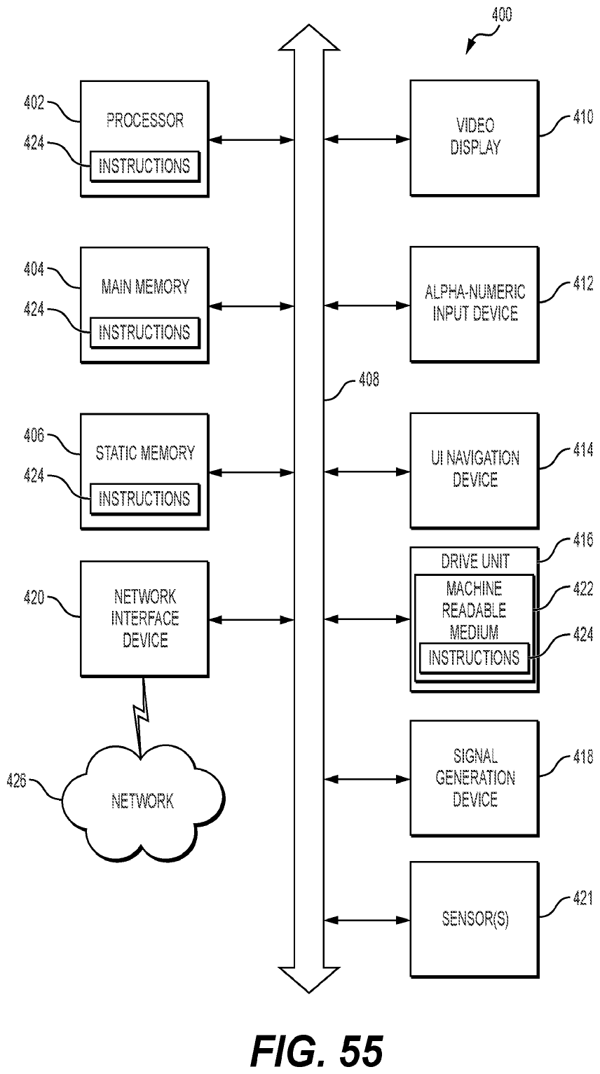

FIG. 55 provides a block diagram illustrating an exemplary machine upon which one or more aspects of embodiments, including methods thereof, herein may be implemented.

Machine 400 may include logic, one or more components, and circuits (e.g., modules). Circuits may be tangible entities configured to perform certain operations. In an example, such circuits may be arranged (e.g., internally or with respect to external entities such as other circuits) in a specified manner. In an example, one or more computer systems (e.g., a standalone, client or server computer system) or one or more hardware processors (processors) may be configured with or by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, may cause the circuit to perform the certain operations.

In an example, a circuit may be implemented mechanically or electronically. For example, a circuit may comprise dedicated circuitry or logic that may be specifically configured to perform one or more techniques such as are discussed above, including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” may be understood to encompass a tangible entity, whether physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiple of such circuits exist contemporaneously, communications may be achieved through signal transmission (e.g., over appropriate circuits and buses) that connect the circuits. In embodiments in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a collection of information.

The various operations of methods described herein may be performed, at least partially, by one or more processors that may temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor-implemented. For example, at least some of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) may be implemented in digital electronic circuitry, in computer hardware, in firmware, in software, or in any combination thereof. Example embodiments may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus can be implemented as, special purpose logic circuitry (e.g., a field programmable gate array (FPGA) or an application-specific integrated circuit (ASIC)).

The computing system or systems herein may include clients and servers. A client and server may generally be remote from each other and generally interact through a communication network. The relationship of client and server arises by virtue of computer programs running on the respective computers and having a client-server relationship to each other. In embodiments deploying a programmable computing system, it will be appreciated that both hardware and software architectures may be adapted, as appropriate. Specifically, it will be appreciated that whether to implement certain functionality in permanently configured hardware (e.g., an ASIC), in temporarily configured hardware (e.g., a combination of software and a programmable processor), or a combination of permanently and temporarily configured hardware may be a function of efficiency. Below are set out hardware (e.g., machine 400) and software architectures that may be implemented in or as example embodiments.

In an example, the machine 400 may operate as a standalone device or the machine 400 may be connected (e.g., networked) to other machines.

In a networked deployment, the machine 400 may operate in the capacity of either a server or a client machine in server-client network environments. In an example, machine 400 may act as a peer machine in peer-to-peer (or other distributed) network environments. The machine 400 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the embodiments discussed herein.

Example machine (e.g., computer system) 400 may include a processor 402 (e.g., a central processing unit (CPU), a graphics processing unit (GPU) or both), a main memory 404 and a static memory 406, some or all of which may communicate with each other via a bus 408. The machine 400 may further include a display unit 410, an alphanumeric input device 412 (e.g., a keyboard), and a user interface (UI) navigation device 411 (e.g., a mouse). In an example, the display unit 410, input device 412 and UI navigation device 414 may be a touch screen display. The machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 may include a machine readable medium 422 on which is stored one or more sets of data structures or instructions 424 (e.g., software) embodying or utilized by any one or more of the methodologies or functions described herein. The instructions 424 may also reside, completely or at least partially, within the main memory 404, within static memory 406, or within the processor 402 during execution thereof by the machine 400. In an example, one or any combination of the processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) that may be configured to store the one or more instructions 424. The term “machine readable medium” may also be taken to include any tangible medium that may be capable of storing, encoding, or carrying instructions for execution by the machine and that cause the machine to perform any one or more of the embodiments of the present disclosure or that may be capable of storing, encoding or carrying data structures utilized by or associated with such instructions. The term “machine readable medium” may accordingly be understood to include, but not be limited to, solid-state memories, and optical and magnetic media. Specific examples of machine readable media may include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” may include any intangible medium that may be capable of storing, encoding or carrying instructions for execution by the machine, and includes digital or analog communications signals or other intangible medium to facilitate communication of such software.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

### REFERENCES

The devices, systems, apparatuses, modules, compositions, computer program products, non-transitory computer readable medium, models, algorithms, and methods of various embodiments disclosed herein may utilize aspects (devices, systems, apparatuses, modules, compositions, computer program products, non-transitory computer readable medium, models, algorithms, and methods) disclosed in the following references, applications, publications and patents and which are hereby incorporated by reference herein in their entirety, and which are not admitted to be prior art with respect to the present embodiments by inclusion in this section:
1. Abdul-Ghani M A, M. Mastsuda, R. Jani, C. P. Jenkinson, D. K. Coletta, K. Kaku, and R. A. DeFronzo, ‘The relationship between fasting hyperglycemia and insulin secretion in subjects with normal or impaired glucose tolearance’, Am. J. Physiol. Endocrinol. Metab., 295(2):E401-406, 2008
2. Ahlqvist E, P. Storm, A. Käräjämäki, M. Martinell, M. Dorkhan, A. Carlsson, et al., ‘Novel subgroups of adult-onset diabetes and their association with outcomes: A data-driven cluster analysis of six variables’, Lancet Diabetes Endocinol, 6:361-369, 2018
3. Amiel S A, Sherwin R S, Simonson D C, Tamborlane W V. 1988. Effect of intensive insulin therapy on glycemic thresholds for counterregulatory hormone release. Diabetes 37: 901-907.
4. Amiel S A, Tamborlane W V, Simonson D C, Sherwin R S. 1997. Defective glucose counterregulation after strict glycemic control of insulin-dependent diabetes mellitus. N Engl J Med 316: 1376-1383.
5. Cobelli C, Dalla Man C, Sparacino G, Magni L, De Nicolao G, Kovatchev B P. 2009. Diabetes: models, signals, and control. IEEE Reviews in Biomedical Engineering 2: 54-96.
6. Cryer P E, Gerich J E. 1985. Glucose counterregulation, hypoglycemia, and intensive therapy of Diabetes Mellitus. N Engl J Med. 313: 232-241.
7. Drucker D J, Nauck M A. 2006. The incretin system: glucagon-like peptide-1 receptor agonists and dipeptidyl peptidase-4 inhibitors in type 2 diabetes. Lancet 368:1696-1705.
8. Kovatchev B P. Diabetes Technology: Monitoring, Analytics, and Optimal Control. Cold Spring Harb Perspect Med 2018 doi: 10.1101/cshperspect.a034389, PMID: 30126835
9. Kovatchev B. A century of diabetes technology: signals, models, and artificial pancreas control. Trends Endocrinol Metab. 2019; 30(7): 432-444. PMID: 31151733
10. Levy J C, D R Matthews, M P Hermans. Correct homeostasis model assessment (HOMA) evaluation uses the computer program; Diabetes Care 1998; 21: 2191-92
11. Matthews D R, J. P. Hosker, A. S. Rudenski, B. A. Naylor, D. F. Treacher & R. C. Turner; Homeostasis model assessment: insulin resistance and β-cell function from fasting plasma glucose and insulin concentrations in man. Diabetologia (1985) 28:412-419
12. Mosteller R D, ‘Simplified calculation of body-surface area’, New England J. Med., 317(17): 1098, 1987
13. Nauck M A, Homberger E, Siegel E G, Allen R C, Eaton R P, Ebert R, Creutzfeldt W. 1986. Incretin effects of increasing glucose loads in man calculated from venous insulin and C-peptide responses. J Clin Endocrinol Metab. 63:492-8.
14. Nauck M, Stockmann F, Ebert R, Creutzfeldt W. 1986. Reduced incretin effect in type 2 (non-insulin-dependent) diabetes. Diabetologia 29:46-52.
15. Van Cauter E, Fabienne Mestrez, Jeppe Sturis and Kenneth S Polonsky, ‘Estimation of insulin secretion rates from C-peptide levels: Comparison of individual and standard kinetic parameters for C-peptide clearance’, Diabetes, 41(3):368-377, 1992
16. Wallace T M, JC Levy, and DR Matthews. Use and Abuse of HOMA Modeling, Diabetes Care 2004 June; 27(6): 1487-1495.
17. White N H, Skor D A, Cryer P E, Levandoski L, Santiago J V. 1983. Identification of Type I Diabetic patients at increased risk for hypoglycemia during intensive therapy. N Engl J Med 308: 485-491.

What is claimed is:

1. A method of predicting one or more subtypes of glucose dysregulation, comprising:
generating, for an in silico population of subjects, modeling for one or more physiological variables which are respectively indicative of the one or more subtypes;
determining whether one or more values of the one or more physiological variables according to the modeling are within a predetermined proximity of one or more respectively corresponding subtype variable values corresponding to the one or more subtypes when the one or more physiological variables are observed in vivo;
in response to the one or more values of the one or more physiological variables according to the modeling being determined to be within the predetermined proximity, fixing one or more parameters defining the modeling; and
applying the modeling, according to the fixed one or more parameters, for a real subject to determine correspondence for the real subject to the one or more subtypes.

2. The method of claim 1, wherein:
the one or more physiological variables comprise one or more of (a) fasting glucose, (b) fasting C-peptide, (c) HOMA2-B, (d) HOMA2-IR, or (e) any combination thereof.

3. The method of claim 1, wherein:
the one or more values of the physiological variables according to the modeling are respectively determined based on one or more of modeling for (l) insulin secretion, (m) C-peptide secretion, (n) β-cell function, (o) insulin resistance, or (p) any combination thereof.

4. The method of claim 1, wherein:
the one or more values of the physiological variables according to the modeling are determined based on β-cell function in dependence on at least a value comprising (ln(fasting C-peptide level))².

5. The method of claim 1, wherein:
the one or more subtypes comprise one or more of (q) SIDD (severe insulin-deficient diabetes), (r) SIRD (severe insulin-resistant diabetes), (s) MOD (mild obesity-related diabetes), (t) MARD (mild age-related diabetes), or (u) any combination thereof.

6. A system for predicting one or more subtypes of glucose dysregulation, comprising:
a processor;
a processor-readable memory including processor-executable instructions for:
generating, for an in silico population of subjects, modeling for one or more physiological variables which are respectively indicative of the one or more subtypes;
determining whether one or more values of the one or more physiological variables according to the modeling are within a predetermined proximity of one or more respectively corresponding subtype variable values corresponding to the one or more subtypes when the one or more physiological variables are observed in vivo;
in response to the one or more values of the one or more physiological variables according to the modeling being determined to be within the predetermined proximity, fixing one or more parameters defining the modeling; and
applying the modeling, according to the fixed one or more parameters, for a real subject to determine correspondence for the real subject to the one or more subtypes.

7. The system of claim 6, wherein:
the one or more physiological variables comprise one or more of (a) fasting glucose, (b) fasting C-peptide, (c) HOMA2-B, (d) HOMA2-IR, or (e) any combination thereof.

8. The system of claim 6, wherein:
the one or more values of the physiological variables according to the modeling are respectively determined based on one or more of modeling for (l) insulin secretion, (m) C-peptide secretion, (n) β-cell function, (o) insulin resistance, or (p) any combination thereof.

9. The system of claim 6, wherein:
the one or more values of the physiological variables according to the modeling are determined based on β-cell function in dependence on at least a value comprising (ln(fasting C-peptide level))².

10. The system of claim 6, wherein:
the one or more subtypes comprise one or more of (q) SIDD (severe insulin-deficient diabetes), (r) SIRD (severe insulin-resistant diabetes), (s) MOD (mild obesity-related diabetes), (t) MARD (mild age-related diabetes), or (u) any combination thereof.

11. A non-transient computer-readable medium having stored thereon computer-readable instructions for predicting one or more subtypes of glucose dysregulation, said instructions comprising instructions causing a computer to:
generate, for an in silico population of subjects, modeling for one or more physiological variables which are respectively indicative of the one or more subtypes;
determine whether one or more values of the one or more physiological variables according to the modeling are within a predetermined proximity of one or more respectively corresponding subtype variable values corresponding to the one or more subtypes when the one or more physiological variables are observed in vivo;
in response to the one or more values of the one or more physiological variables according to the modeling being determined to be within the predetermined proximity, fix one or more parameters defining the modeling; and
apply the modeling, according to the fixed one or more parameters, for a real subject to determine correspondence for the real subject to the one or more subtypes.

12. The medium of claim 10, wherein:
the one or more physiological variables comprise one or more of (a) fasting glucose, (b) fasting C-peptide, (c) HOMA2-B, (d) HOMA2-IR, or (e) any combination thereof.

13. The medium of claim 10, wherein:
the one or more values of the physiological variables according to the modeling are respectively determined based on one or more of modeling for (l) insulin secretion, (m) C-peptide secretion, (n) β-cell function, (o) insulin resistance, or (p) any combination thereof.

14. The medium of claim 10, wherein:
the one or more values of the physiological variables according to the modeling are determined based on β-cell function in dependence on at least a value comprising (ln(fasting C-peptide level))².

15. The medium of claim 10, wherein:
the one or more subtypes comprise one or more of (q) SIDD (severe insulin-deficient diabetes), (r) SIRD (severe insulin-resistant diabetes), (s) MOD (mild obesity-related diabetes), (t) MARD (mild age-related diabetes), or (u) any combination thereof.
